SEQUENCE LISTING (55 sequences)

SEQ ID NO: 1 - protein, 109 aa, Homo sapiens
TLERNHVNVNSVVNPLVILLPIEYIKELTLEKSLMNIRNVGKHFIVPDPIVDMKGFTWEKRLINVRNVEKHSRVPVMFVYMKGPTLGKISMNVSSVGKHYPLLQVFKHT

SEQ ID NO: 2 - protein, 56 aa, Homo sapiens
GKVDVTSTQKEAENQRRVVTGSVSSSRSSEMSSSKDRPLSARERRRQACGRTRVTS

SEQ ID NO: 3 - protein, 67 aa, homo sapiens
SRRNQNCATEIPQIVEISIEKDNDSCVTPGTRLARRDSYSRHAPWGGKKKHSCSTKTQSSLDADKKF

SEQ ID NO: 4 - protein, 56 aa, homo sapiens
RNTILRQARNHKLRVDKAAAAAAALQAKSDEKAAVAGKKPVVGKKGKACGRTRVTS

SEQ ID NO: 5 - protein, 86 aa, homo sapiens
YWVGEDSTYKFFEVILIDPFHKAIRRNPDTQWITKPVHKHREMRGLTSAGRKSRGLGKGHKFHHTIGGSRRAAWRRRNTLQLHRYR

SEQ ID NO: 6 - protein, 70 aa, homo sapiens
KLLSISGKRSAPGGGSKVPQKKVKLAADEDDDDDDEEDDDEDDDDDDFDDEEAEEKAPVKKSIRDTPAKN

SEQ ID NO: 7 - protein, 141 aa, homo sapiens
  misc_feature (116)..(116): X may be any naturally occurring amino acid
NKPAVTTKSPAVKPAAAPKQPVGGGQKLLTRKADSSSSEEESSSSEEEKTKKMVATTKPKATAKAALSLPAKQAPQGSRDSSSDSDSSSSEEEEEKTSKSAVKKKPQKVAGGAAPXKPASAKKGKAESSNSSSSDDSSEEE

SEQ ID NO: 8 - protein, 84 aa, homo sapiens
ASFPQHHHPGIPGVAHSVISTRTPPPPSPLPFPTQAILPPAPSSYFSHPTIRYPPHLNPQDTLKNYVPSYDPSSPQTSQSWYLG

SEQ ID NO: 9 - protein, 84 aa, homo sapiens
PKRRSARLSAKPPAKVEAKPKKAAAKDKSSDKKVQTKGKRGAKGKQAEVANQETKEDLPAENGETKTEESPASDEAGEKEAKSD

SEQ ID NO: 10 - protein, 63 aa, homo sapiens
AMFFIGFTALVIMWQKHYVYGPLPQSFDKEWVAKQTKRMLDMKVNPIQGLASKWDYEKNEWKK

SEQ ID NO: 11 - protein, 68 aa, homo sapiens
ATKKKSKDKEKDRERKSESDKDVKVTRDYDEEEQGYDSEKEKKEEKKPIETGSPKTKECSVEKGTGDS

SEQ ID NO: 12 - protein, 106 aa, homo sapiens
ESFKRLVTPRKKSKSKLEEKSEDSIAGSGVEHSTPDTEPGKEESWVSIKKFIPGRRKKRPDGKQEQAPVEDAGPTGANEDDSDVPAVVPLSEYDAVEREKLAAALE

SEQ ID NO: 13 - protein, 85 aa, homo sapiens [sequence omitted]

SEQ ID NO: 14 - protein, 97 aa, homo sapiens
ARGSEFKSPEQFSDEVEPATPEEGEPATQRQDPAAAQEGEDEGASAGQGPKPEAHSQEQGHPQTGCECEDGPDGQEMDPPNPEEVKTPEEGEKQSQC

SEQ ID NO: 15 - protein, 116 aa, homo sapiens
ARGSEFKHGTVELQGSQTALYRTGSVGIGNLQRTSSQRSTLTYQRNNYALNTTATYAEPYRPIQYRVQECNYNRLQHAVPADDGTTRSPSIDSIQDHARQTPWGPSEACGRTRVTS

SEQ ID NO: 16 - protein, 111 aa, homo sapiens
LAFVPISGWNGDNMLEPSANMPWFKGWKVTRKDGNASGTTLLEALDCILPPTRPTDKPLRLPLQDVYKIGGIGTVPVGRVETGVLKPGMVVTFAPVNVTTEVKSVEMHHEA

SEQ ID NO: 17 - protein, 19 aa, homo sapiens
MLGDPNSSISLKFQAMDVG

SEQ ID NO: 18 - protein, 37 aa, homo sapiens
ARGSEFKHLIEVSGNGCGVEEENFEGLISFSSETSHI

SEQ ID NO: 19 - protein, 58 aa, homo sapiens
LGDRTLGPKVHTLHSLVKTRRPGNKKGSPNTAVYKTVLVSYEVKEGESQSCSQFTCLC

SEQ ID NO: 20 - protein, 143 aa, homo sapiens
  misc_feature (51)..(51): X may be any naturally occurring amino acid
ARGSEFKLLLKVIILGDSGVGKTSLMNQYVNKKFSNQYKATIGADFLTKEXMVDDRLVTMQIWDTAGQERFQSLGVAFYRGADCCVLVFDVTAPNTFKTLDSWRDEFLIQASPRDPENFPLVCFRGQSCFPTQQACGRTRVTS

SEQ ID NO: 21 - protein, 77 aa, homo sapiens
  misc_feature (5)..(5): X may be any naturally occurring amino acid
CSGTXTISDIAGQPGPLMPCMHLRPFXGQLVKQMLDDFXXHRYIANLGHGLYPDMDPEHVGAFVDAVHKHSRLLRQN

SEQ ID NO: 22 - protein, 85 aa, homo sapiens [sequence omitted]

SEQ ID NO: 23 - protein, 97 aa, homo sapiens; identical to SEQ ID NO: 14

SEQ ID NO: 24 - DNA, 301 bp, homo sapiens
  misc_feature (70)..(70): n is a, c, g, or t
tccggggacgaattcctggtagcctcattcagccgatggaaggtagaagggactcagaacttcaggcctnattctgcgtttttgtatgccccaagaatgaaagggctctttgtgaatttgcatgtagatttatttaacattcaaccggcagaaaacggaaggtagtgcatgacactggggggaaccaggcccccgcccacctcacatcgtcatggcattagctgtttactggctcccgtggaaacattggaaggggatttgttttgtggttgggtttccttttttttttttttttaaccag

SEQ ID NO: 25 - DNA, 354 bp, homo sapiens
  misc_feature (332)..(332): n is a, c, g, or t
gattcttcctacctttgtcagctactgagttgcttctggggagggaagtacttccttgcccctccccaacccccctacctcaccatatcctatcatatcttgatagtcatggggaagaggatgtgcacacagacatacaaatttcctcaaagctggagagaccaggctacatgtgagctcatagatgctgctgaggctcatcctgagggctggatggttggccagggtttcagaatgagggtaagggatgagcactgccacccaagcttgcggccgcactcgagtaactagttaaccccttggggcctctaaacgggtcttgaggggttaantagtgactcgagtgcggccgca

SEQ ID NO: 26 - DNA, 533 bp, homo sapiens
  misc_feature (433)..(433): n is a, c, g, or t
atgctcggggatccgaattcaagcatctcattgaagtttcaggcaatggatgtggggtagaagaagaaaacttcgaaggcttaatctctttcagctctgaaacatcacacatctaagattcgagagtttgccgacctaactcgggttgaaacttttggctttcaggggaaagctctgagctcactttgtgcactgagtgatgtcaccatttctacctgccacgtatcggcgaaggttgggactcgactggtgtttgatcacgatgggaaaatcatccagaaaaccccctacccccaccccagagggaccacagtcagcgtgaagcagttattttctacgctacctgtgcgccataaggaatttcaaaggaatattaagaagtacagaacctgctaaggccatcaaacctattgatcggaagtcagtccatcanatttgctctgggccggtggtactgagtctaagcactgcggtgaagaagatagtaggaaacagtctggatgctggtgccactaatattgatctaaagcttg

SEQ ID NO: 27 - DNA, 455 bp, homo sapiens
  misc_feature (410)..(410): n is a, c, g, or t
gggacgattagctagcatttgtgccaatttctggttggaatggtgacaacatgctggagccaagtgctaacatgccttggttcaagggatggaaagtcacccgtaaggatggcaatgccagtggaaccacgctgcttgaggctctggactgcatcctaccaccaactcgtccaactgacaagcccttgcgcctgcctctccaggatgtctacaaaattggtggtattggtactgttcctgttggccgagtggagactggtgttctcaaacccggtatggtggtcacctttgctccagtcaacgttacaacggaagtaaaatctgtcgaaatgcaccatgaagcttgcggccgcactcgagtaactagttaaccccttggggcctctaaacgggtcttggaggggttaacnagttgctcgagtggggcggcnggctncttggtggtttatttcaga

SEQ ID NO: 28 - DNA, 523 bp, homo sapiens
ctcggggatccgaatttcaagcggcaagaagtttcagaataagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagcttgcggccgcactcgagtaactagttaaccccttggggcctctaaacgggtcttgaggggttaactcgagttactcgtgggcgcagctctttgcttagtatttttaatggttggttgtaacctttcgtttctcatcgccgaattatgatggttttaaataatgatcataattctttctttttacttggtttttttttttcacttttactttctgtttatgaagcacgcccgccccacaa

SEQ ID NO: 29 - DNA, 418 bp, homo sapiens
atgctcggggatccgaattcagcttgggaacgcggccatttcaaaggggaagccaaaatctcaagaaattcccagcaggttacctggaggcggatcatctaattctctgtggaatgaatacacacatatatattacaagggataagcttgcggccgcactcgagtaactagttaaccccttggggcctctaaacgggacttgaggggtaagctagttactcgagggcgagcttatgggaaatatatattgcggtatttaaggaattagttacccgctcgctggcctttgaactgttgtttgaggccttaaattgatgatcgtggtgggaaacaagaggtggggtgggagatttgttttttgttctgaagcggggaggggactagaccctaaaagcatttaaatataagacaacccaat

SEQ ID NO: 30 - DNA, 416 bp, homo sapiens
  misc_feature (233)..(233): n is a, c, g, or t
gggacgatcagcattgaatgaatgttggctacaaaatcaattcttggtgttgtatcagaggagtaggagagaggaaacatttgacttatctggaaaagcaaaatgtacttaagaataagaataacatggtccattcacctttatgttatagatatgtctttgtgtaaatcatttgttttgagttttcaaagaatagcccattgttcattcttgtgctgtacaatgaccactgnttattgttactttgacttttcagagcacacccttcctctggtttttgtatatttattgatggatcaataataatgaggaaagcatgatatgtatattgctgagttgttagccttttaagcttgcggccgcactcgagtaactagttaaccccttggggcctctaaacgggtcttgaggggtta

SEQ ID NO: 31 - DNA, 499 bp, homo sapiens
  misc_feature (18)..(18): n is a, c, g, or t
ggtacgaattagccaganatcggggcgagtacaatggggatgtgggcgcgggagccccgctccccttttttagcagcacctcccagccccgcagaataaaaccgatcgcnncccctccgcgcgcgccctcccccgagatgcggagcgggaggaggcggcggcggccgaggaggaggaggaggaggccccggaggaggaggcgttggaggtcgaggcggaggcggaggaggaggaggccgaggcgccggangaggccnaggcgccggagcaggaggaggccggccggaggcggcatgagacgagcgtggcggccgcggctgctcggggccgcgctggttgcccattgacagcggcgtctgcagctcgcttcaagatggccgcttggctcgcattcattttctgctgaacgacttttaactttcnttgtcttttccgcccgcttcnatcgcctcncgccggctgctctttccgggattttttatcaagcagaaatgcatcg

SEQ ID NO: 32 - DNA, 21 bp, homo sapiens
attgtgaataagcataaggtt

SEQ ID NO: 33 - DNA, 21 bp, homo sapiens
gagcggtctctgagtccgatt

SEQ ID NO: 34 - DNA, 21 bp, homo sapiens
ttgagtcagaatccgcataag

SEQ ID NO: 35 - DNA, 21 bp, homo sapiens
aatgcgagtcataagtgttct

SEQ ID NO: 36 - DNA, 21 bp, homo sapiens
aatgcgctggctaatccttcg

SEQ ID NO: 37 - DNA, 21 bp, homo sapiens
gcgaagccgccgaagctgtct

SEQ ID NO: 38 - DNA, 21 bp, homo sapiens
agggctctggatccggattcg

SEQ ID NO: 39 - DNA, 21 bp, homo sapiens
atactactgggtcgcctctgt

SEQ ID NO: 40 - DNA, 21 bp, homo sapiens
aaggttaatactcatcatact

SEQ ID NO: 41 - DNA, 21 bp, homo sapiens
ctgtttctgacggcgcaggcg

SEQ ID NO: 42 - DNA, 21 bp, homo sapiens
tttaattggtataattcgtcg

SEQ ID NO: 43 - DNA, 21 bp, homo sapiens
cttccgcatcagctgcggtgg

SEQ ID NO: 44 - DNA, 21 bp, homo sapiens
cttgcgtggtatgcgaagagt

SEQ ID NO: 45 - DNA, 21 bp, homo sapiens
aagattgggacggcgtggctt

SEQ ID NO: 46 - DNA, 21 bp, homo sapiens
acgcctactcatggtgggaag

SEQ ID NO: 47 - DNA, 21 bp, homo sapiens
actcctacttatgcggggtat

SEQ ID NO: 48 - DNA, 21 bp, homo sapiens
atgccggctactacgcctcag

SEQ ID NO: 49 - DNA, 21 bp, homo sapiens
aaggcgtggtttgggcagatt

SEQ ID NO: 50 - DNA, 21 bp, homo sapiens
aagaattggtttggtcatacg

SEQ ID NO: 51 - DNA, 21 bp, homo sapiens
catactcatcatgataagcat

SEQ ID NO: 52 - DNA, 21 bp, homo sapiens
attacgaataagtgggggtat

SEQ ID NO: 53 - DNA, 21 bp, homo sapiens
ctgaatacgcattcgtctcag

SEQ ID NO: 54 - DNA, 21 bp, homo sapiens
gggcctgcgtgggaggatccg

SEQ ID NO: 55 - DNA, 27 bp, homo sapiens
agtcagtcttatcataagcgtactagc

(19) United States
(12) Patent Application Publication
(10) Pub. No.: US 2014/0066329 A1
(43) Pub. Date: Mar. 6, 2014

(54) LUNG CANCER DIAGNOSTIC ASSAY

(71) Applicant: University of Kentucky Research Foundation, Lexington, KY (US)

(72) Inventors: Edward A. Hirschowitz, Lexington, KY (US); Li Zhong, Walnut, CA (US); Arnold J. Stromberg, Lexington, KY (US); Nada H. Khattar, Lexington, KY (US)

(73) Assignee: University of Kentucky Research Foundation, Lexington, KY (US)

(21) Appl. No.: 14/046,271

(22) Filed: Oct. 4, 2013

Related U.S. Application Data
(63) Continuation of application No. 13/430,474, filed on Mar. 26, 2012, now abandoned.

(51) Int. Cl. G01N 33/574

(52) U.S. Cl. CPC G01N 33/57423
USPC 506/9

(57) ABSTRACT

A method for selecting a person at risk for lung cancer to undergo radiographic testing is provided. The method provides for the identification of markers for lung cancer in a population of patients that have not previously diagnosed with the disease. The markers identify autoantibodies present in a fluid sample of a patient who may not show other symptoms of lung cancer.

### CROSS REFERENCE TO RELATED APPLICATIONS

This application is a 35 U.S.C. §120 continuation of co-pending U.S. patent application Ser. No. 13/430,474, filed Mar. 26, 2012, which is a 35 U.S.C. §120 continuation of U.S. patent application Ser. No. 12/093,082, filed Aug. 19, 2008, now abandoned, which is a 35 U.S.C. §371 National Stage Application of PCT application number PCT/US2006/060796, filed Nov. 10, 2006, and which claims priority from U.S. Patent Application Ser. Nos. 60/735,555, filed on Nov. 10, 2005, 60/735,418, filed on Nov. 10, 2005, and 60/806,778, filed Jul. 2, 2006, from which this application also claims priority. All the applications listed above are incorporated herein by reference in their entireties.

### BACKGROUND

Lung cancer is the leading cause of cancer death for both men and women in the United States and many other nations. The number of deaths from this disease has risen annually over the past five years to nearly 164,000 in the U.S. alone, the majority succumbing to non-small cell cancers (NSCLC). This exceeds the death rates of breast, prostate and colorectal cancer combined.

Many experts believe that early detection of lung cancer is a key to improving survival. Studies indicate that when the disease is detected in an early, localized stage and can be removed surgically, the five-year survival rate can reach 85%. But the survival rate declines dramatically after the cancer has spread to other organs, especially to distant sites, whereupon as few as 2% of patients survive five years. Unfortunately, lung cancer is a heterogeneous disease and is usually asymptomatic until it has reached an advanced stage. Thus, only 15% of lung cancers are found at an early, localized stage. There is, therefore, a compelling need for tools that aid in the screening of asymptomatic persons leading to detection of lung cancer in its earliest, most treatable stages.

Chest X-ray and computed tomography (CT) scanning have been studied as potential screening tools to detect early stage lung cancer. Unfortunately, the high cost and high rate of false positives render these radiographic tools impractical for widespread use. For example, a recent study of the U.S. National Cancer Institute concluded that screening for lung cancer with chest X-rays can detect early lung cancer but produces many false-positive test results, causing needless follow-up testing, Oken et al, Journal of the National Cancer Institute, 97(24) 1832-1839, 2005. Of the 67,000 patients who received a baseline X-ray on entering the trial, nearly 6,000 (9%) had abnormal results that required follow-up. Of these, only 126 (2% of the 6,000 participants with abnormal X-rays) were diagnosed with lung cancer within 12 months of the initial chest X-ray.

A similar problem with false positives is being encountered with ongoing trials involving CT scans. Specificity of CT screening is calculated at around 65% based on the number of indeterminate radiographic findings.

Experts raise serious concerns about health cost per life saved when assessing the number of cancers detected per number of CT screening scans performed because a large portion of the incurred health care costs can be attributed to the number of indeterminate pulmonary nodules found on prevalence scanning that require further investigation, many of which ultimately are found to be benign.

PET scans are another diagnostic option, but PET scan are costly, and generally not amenable for use in screening programs.

Currently, age and smoking history are the only two risk factors that have been used as selection criteria by the large screening studies.

A blood test that could detect radio graphically apparent cancers (>0.5 cm) as well as occult and pre-malignant cancer (below the limit of radiographic detection) would identify individuals for whom radiologic screening is most warranted and de facto would reduce the number of benign pulmonary findings that require further workup.

It is clear, therefore, there is an urgent need for improved lung cancer screening and detection tools that overcome the aforementioned limitations of radiographic techniques.

### SUMMARY

The present invention relates to assays, methods, and kits for the early detection of lung cancer using body fluid samples. In particular, the invention relates to detection of lung cancer by evaluating the presence of one or a panel of markers, such as autoantibody biomarkers.

The present invention may be employed in a comprehensive lung cancer screening strategy especially when used in concert with radiographic imaging and other screening modalities. The present invention can be used to enrich the population for further radiographic analysis to rule out the possible presence of lung cancer.

In short, the invention is directed to a method of detecting the probable presence of lung cancer in a patient, in one embodiment, by providing a blood sample from the patient and analyzing the patient blood sample for the presence of one or a panel of autoantibodies associated with lung cancer. The panel can be identified, for example, by assessing the maximum likelihood of cancer associated with the members of the panel. Any of a variety of statistical tools can be used to assess the simultaneous contribution of multiple variables to an outcome.

The present invention was employed to analyze samples obtained during a major CT screening trial and to distinguish early and late stage lung cancer as well as occult disease from risk-matched controls. The instant assay predicted with almost 90% accuracy the presence of lung cancer as many as five years prior to radiographic detection. The instant assay can be used as a screening test for asymptomatic patients, or patients of a high risk group which have not yet been diagnosed with lung cancer using acceptable tests and protocols, that is, for example, they lack radiographically detectable lung cancer.

The invention provides an alternative to the high cost and low specificity of current lung cancer screening methods, such as chest X-ray or Low Dose CT. The instant assay maximizes cancer detection rates while limiting the detection of benign pulmonary nodules that could require further evaluation and therefore, is a powerful and cost effective tool that can be readily incorporated into a comprehensive early detection strategy.

These and other features, aspects, and advantages of the present invention will become better understood with regard to the following description and appended claims.

### DETAILED DESCRIPTION

Early diagnosis of pathologic states is beneficial. However, not all pathologic states have readily detectable, simple signatures. Other pathologic states are heterogeneous in etiology or phenotype, or throughout the developmental stage thereof. In such circumstances, a single, sensitive and specific diagnostic signature or marker is unlikely to exist.

Nevertheless, it now is possible to develop a suitable diagnostic assay using a plurality of markers, that alone may not have sufficient predictive power, but in certain combination, a panel has sufficient specificity and sensitivity for practical use. Moreover, multiplex techniques and data handling capacity enable the flexibility of developing particularized and personalized diagnostic assays with ease of use and greater predictive power for defined populations or for the general population.

The present invention provides a new assay and method for detecting disease, such as, lung cancer, earlier and more accurately than conventional means. In short, a sample from the patient or subject, such as a blood sample, is obtained and is analyzed for the presence or absence of a panel of antibody biomarkers. For lung cancer, one or a panel of markers is used, each marker associated to some degree with lung cancer, and the majority of which when a panel is used yields a predictable measure of the likelihood of having lung cancer in a heterogeneous population.

As set forth in more detail below, the assay and method according to the present invention correctly identified patients with early and late stage lung cancer. Identification of patients with early stage lung cancer is particularly valuable as current assays and screening modalities have little ability to do so in a robust and cost effective fashion. The instant screening assay provides greater predictability and produces fewer false positives than assays currently used, which often are costly as well. The instant assay also is versatile, by using an assay format that enables testing a large number of samples simultaneously, such as using a microarray, control samples relative to any population can be run in parallel to obtain discriminating data of high confidence, wherein the plurality of controls are matched for as many parameters as possible to the test population. That enables correction for population differences, such as race, sex, age, polymorphism and so on that may arise and could confound results.

### DEFINITIONS

As used herein, the following terms shall have the following meanings

“Lung cancer” means a malignant process, state and tissue in the lung.

“Protein” is a peptide, oligopeptide or polypeptide, the terms are used interchangeably herein, which is a polymer of amino acids. In the context of a library, the polypeptide need not encode a molecule with biologic activity. An antibody of interest binds an epitope or determinant. Epitopes are portions of an intact functional molecule, and in the context of a protein, can comprise as few as about three to about five contiguous amino acids.

“Normalized” relates to a statistical treatment of a metric or measure to correct or adjust for background and random contributions to the observed result to determine whether the metric, statistic or measure is a true reflection, response or result of a reaction or is non-significant and random.

“Non-Small Cell Lung Cancer” (NSCLC) is a subtype of lung cancer that accounts for about 80% of all lung cancers, as compared to small cell cancer which is characterized by small, ovoid cells, also known as oat cell cancer. Included in the NSCLC subtype are squamous cell carcinoma, adenocarcinoma and large cell carcinoma.

“Body fluid” is any liquid sample obtained or derived from a body, such as blood, saliva, semen, tears, tissue extracts, exudates, body cavity wash, serum, plasma, tissue fluid and the like that can be used as a patient sample for testing. Preferably the fluid can be used as is, however, treatment, such as clarification, for example, by centrifugation, can be used prior to testing. A sample of a body fluid is a fluid sample.

“Blood sample” means a small aliquot of, generally, venous blood obtained from an individual. The blood can be processed, for example, clotting factors are inactivated, such as with heparin or EDTA, and the red blood cells are removed to yield a plasma sample. The blood can be allowed to clot, and the solid and liquid phases separated to yield serum. All such “processed” blood samples fall within the scope of the definition of “blood sample” as used herein.

“Epitope” means that particular molecular structure bound by an antibody. A synonym is “determinant.” A polypeptide epitope may be as small as 3-5 amino acids.

“Biomarker” denotes a factor, indicator, score, metric, mathematic manipulation and the like that is evaluated and found to be useful in predicting an outcome, such as the current status or a future health status in a biological entity. A biomarker is synonymous with a marker. “Panel” means a compiled set of markers that are measured together for an in an assay. A panel can comprise 2 markers, 3 markers, 4 markers, 5 markers, 6 markers, 7 markers, 8 markers, 9 markers, 10 markers, 11 markers, 12 markers or more. The statistical treatment and the assay methods taught in the instant application and which can be applied in the practice of the instant invention provide for use of any of a number of informative markers in an assay of interest.

“Outcome” is that which is predicted or detected.

“Autoantibodies” mean immunoglobulins or antibodies (the terms are used interchangeably herein) directed to “autologous” (self) proteins including pathologic cells, such as infected cells and tumor cells. In this case, antibodies against tumor are derived from an individual's own tumor, which is a genetic aberration of his/her own cells.

“Weighted sum” means a compilation of scores from individual markers, each with a predictive value. Markers with greater predictive value contribute more to the sum. The relative value of the individual markers is derived statistically to maximize the value of a multivariable expression, using known statistical paradigms, such as logistic regression. A number of commercially available statistics packages can be used. In a formula, such as a regression equation, of additive factors, the “weight” of each factor (marker) is revealed as the coefficient of that factor.

“Statistically significant” means differences unlikely to be related to chance alone.

“Marker” is a factor, indicator, metric, score, mathematic manipulation and the like that is evaluated and usable in a diagnosis. A marker can be, for example, a polypeptide or an antigen, or can be an antibody that binds an antigen. A marker also can be any one of a binding pair or binding partners, a binding pair or binding partners being entities with a specificity for one another, such as an antibody and antigen, hormone and receptor, a ligand and the molecule to which the ligand binds to form a complex, an enzyme and co-enzyme, an enzyme and substrate and so on.

“Forecast marker” is a marker that is present before detection of lung cancer using known techniques. Thus, the instant assay detects lung cancer-specific autoantibodies prior to a radiographically detectable cancer is found in a patient, for example, up to five years before a radiographically detectable cancer is noted. Such autoantibodies are forecast markers.

“Target population” means any subset of a population typified by a particular marker, state, condition, disease and so on. Thus, the target population can be particular patients with a particular form or stage of lung cancer, or a population of smokers, for example. A target population may comprise people with one or more risk factors. A target population may comprise people with a suspect test result, such as presence of an abnormality in the lung deserving of further and more timely monitoring.

“Radiographic” refers to any imaging method, such as CAT, PET, X-ray and so on.

“Radiographically detectable cancer” refers to diagnosing or detection of cancer by a radiographic means. The presence of cancer generally is confirmed by histology.

“Tissue sample” refers to a sample from a particular tissue. For a tissue sample that is in liquid form, the sample can be a body fluid or can come from a liquid tissue, such as blood, or a processed blood aliquot. The phrase also relates to a fluid obtained from a solid tissue, such as, for example, an exudate, spent tissue culture fluid, the washings of a minced solid tissue and so on.

### Biomarker Selection

The selection and identification of lung cancer associated markers, such as, autoantibodies, and the proteins having specific affinity thereto or are bound thereby, can be by any means using methods available to the artisan. In the case of antibody biomarkers, any of a variety of immunology-based methods can be practiced. As known in the art, aptamers, spiegelmers and the like which have a binding specificity also can be used in place of antibody. Many known high throughput methods relying on an antibody-antigen reaction can be practiced in the instant invention.

Molecules from individuals in the target population can be compared to those from a control population to identify any which are lung cancer-specific, using, for example, subtraction selection and so on. Alternatively, the target population and normal (control) population samples can be used to identify molecules which are specific for the target population from a library of molecules.

A form of affinity selection can be practiced with libraries, using an antibody as probe to screen a library of candidate molecules. The use of an antibody to screen the candidates is known as “biopanning” Then it remains to validate the target population-specific molecules and the use thereof, and then to determine the power of the individual markers as predictors of members of the target population.

A suitable means is to obtain libraries of molecules, whether specific for lung cancer or not, and to screen those libraries for molecules that bind antibodies in members of the target population. Because protein or polypeptide epitopes can be as small as 3 amino acids, but can be less than 10 amino acids in length, less than 20 amino acids in length and so on, the average size of the individual members of the library is a design choice. Thus, smaller members of the library can be about 3-5 amino acids to mimic a single determinant, whereas members of 20 or more amino acids may mimic or contain 2 or more determinants. The library also need not be restricted to polypeptides as other molecules, such as carbohydrates, lipids, nucleic acids and combinations thereof, can be epitopes and thus be used as or to identify markers of lung cancer.

Because the biomarker identification process seeks to identify epitopes rather than intact proteins or other molecules, the scanned or screened libraries need not be lung cancer-specific but can be obtained from molecules of normal individuals, or can be obtained from populations of random molecules, although use of samples from lung cancer patients may enhance the likelihood of identifying suitable lung cancer biomarkers. The epitopes, or cross-reactive molecules, nevertheless, are present and are immunogenic in patients with lung cancer, irrespective of the function of the molecules containing the epitopes.

Exemplifications of those methods are described in the Examples using T7 lung cancer-specific cDNA phage libraries and an M 13 random peptide library. Both were carried in phage display libraries, as known in the art. One of the T7 phage NSCLC cDNA libraries used was commercially available (Novagen, Madison, Wis., USA), and the other T7 library was constructed from the adenocarcinoma cell line, NCI-1650 (gift of H. Oie, NCI, National Institutes of Health, Bethesda, Md., USA).

Thus, a phage library can be constructed as known in the art. Total RNA from target tissue or cells is extracted and selected. First-strand cDNA synthesis is conducted, ensuring representation of both N-terminal and C-terminal amino acid sequences. The cDNA product is ligated into a compatible phage vector to generate the library. The library is amplified in a suitable bacterial host and for lytic phage, such as T7, the cells are lysed to obtain a phage prep.

Lysates are titered under standard conditions and stored after purification. For other phage, virus may be shed into the medium, such as with M1 3, in which case virus is collected from the supernatant and titered.

The phage library is biopanned or screened with a tissue sample, preferably a fluid sample, such as a plasma or serum, from patients with lung cancer, and with an analogous tissue sample, such as plasma or serum from normal healthy donors, to identify potential displayed molecules recognized by ligands, such as circulating antibodies, in patients with lung cancer.

In one embodiment, the tissue sample is a blood sample, such as plasma or serum, and the goal is to identify markers recognized by antibodies found in the plasma or serum of the target population, such as, non-small cell lung cancer patients. To remove phages that are recognized by antibodies of the non-target population from the library, the phage display library is, for example, exposed to normal serum or pooled sera. Unreacted phages are separated from those reacting with the non-target population samples. The unreacted phages then are exposed to NSCLC serum to isolate phages recognized by antibodies in the sera of patients with NSCLC. The reactive phage are collected, amplified in a suitable bacteria host, the lysates are collected, stored, and are identified as “sample 1” or as “biopan 1.” The biopan and amplification processes can be repeated multiple times, generally using the same control and target samples to enhance the purification process.

Phages from the biopans represent an enriched population that is more likely to contain expressed molecules recognized specifically by antibodies in samples from NSCLC patients. As many phage libraries express polypeptides, the selected phages can be said to express and to represent “capture peptides” for NSCLC associated antibodies.

To further select phage clones that express molecules that are bound byNSCLC-specific antibodies, individual phage lysates selected in the biopans can be robotically spotted on, for example, slides (Schleicher and Schuell, Keene, N. H.) using an Arrayer (Affymetrix, Santa Clara, Calif.) to produce a microarray with a plurality of candidate phage-expressed molecules which were bound by antibodies in the sera of NSCLC patients.

To identify which phage display molecules are likely to be NSCLC-specific capture molecules (able to bind NSCLC-specific antibodies), the screening slide is incubated with, for example, individual NSCLC patient serum samples, ideally, not those used in the biopans, and further screened using standard immunoassay methodology. Antibodies bound to phages can be identified, for example, by dual color labeling with suitable immune reagents, as known in the art, wherein phage vector expression product is labeled with a first colored or detectable reporter molecule, to account for the amount of expression product at each site, and antibody bound to the phage expressed polypeptide is labeled with a second colored or detectable reporter molecule, distinguishable from the first reporter molecule.

One convenient way of interpreting the data for identifying the capture molecules associated or specific for NSCLC bound by antibodies in NSCLC samples is by computer-assisted regression analysis of multiple variables that indicates the mean signal and standard deviation of all polypeptides on the slide. The statistical treatment is directed at an individual phage to determine specificity, and also is directed at a plurality of phage to determine if a subset of phage can provide greater predictive power of determining whether a sample is from a patient with or is likely to have NSCLC. The statistical treatment of monitoring plural samples enables determining the level of variability within an assay. As the populations sampling increases, the variability can be used to assess between assay variability and provide reliable population parameters.

Thus, phages that bind antibodies in patient samples to a greater degree than other phage on the slide, chip and so on, are considered candidates, when, for example, the signal is >1, >2, >3 or more standard deviations from the norm (the mean signal on the chip). In some of the experiments described herein, the candidates represented about 1/100 of the phage display polypeptides on the screening chip constructed with a T7 library biopanned four times.

The candidate phage clones are compiled on a “diagnostic chip” and further evaluated for independent predictive value in discriminating samples of NSCLC patients from samples of a non-NSCLC population.

Diagnostic markers are selected for the ability to signal/detect/identify the presence of or future presence of radio logically detectable lung cancer in a subject. As some conditions have multiple etiologies, multiple cellular origins and so on, and with any disease, is presented on a heterogeneous background, a panel or plurality of markers may be more predictive or diagnostic of that particular condition. Lung cancer is one such condition.

As known in the biostatistic arts, there are a number of different statistical schemes that can be implemented to ascertain the collective predictive power of related multiple variables, such as a panel of markers or reactivity with a panel of markers. Thus, for example, a dynamic statistical modeling can be used to interpret data from a plurality of factors to develop a prognostic test relying on the use of two or more of such factors. Other methods include Bayesian modeling using conditional probabilities, least squares analysis, partial least squares analysis, logistic multiple regression, neural networks, discriminant analysis, distribution-free ranked-based analysis, combinations thereof, variations thereof and so on to select a panel of suitable markers for inclusion in a diagnostic assay. The goal is the handling of multiple variables, and then to process the data to maximize a desired metric, see for example, Pepe & Thompson, Biostatistics 1, 123-140, 2000; McIntosh & Pepe, Biometrics 58, 657-664, 2002; Baker, Biometrics 56, 1082-1087, 2000; DeLong et al, Biometrics AA, 837-845, 1988; and Kendziorski et al., Biometrics 62, 19-27, 2006, for example.

Hence, in certain circumstances, the statistical treatment seeks to maximize a predictive metric, such as the area under the curve (AUC) of receiver operating characteristic (ROC) curves. The treatments yield a formulaic approach or algorithm to maximize outcomes relying on a selected set of variables, revealing the relative influence of any one or all of the variables to the maximized outcome. The relative influence of a marker can be viewed in a derived formula describing the relationship as a coefficient of a variable. Thus, for example, the two panels of five markers identified in the exemplified studies described hereinbelow were selected from such an analysis, and the maximal AUC, a score, is described by a formula including the five markers, with the relative weight of any one marker in the formula to obtain maximal predictive power represented as a coefficient of that any one variable. The coefficient represents a weighting, and the derived formula can be viewed as a sum of weighted variables yielding a weighted sum.

The goal is to find a balance in maximizing, for example, specificity and sensitivity, or the positive predictive value, over a selected, and preferentially, minimal plurality of variables (the markers) to enable a robust diagnostic assay in light of those parameters. The weight or influence of a variable to the maximized outcome is derived from the data so far ascertained and analyzed, and recalculated as the number of patients analyzed increases. As the number of patients increases, so can the confidence that a metric represents a population mean value with a confidence limit range of values about the mean.

As noted in the examples hereinbelow, the exemplified five marker panels contain markers which have individual specificity that exceeds the observed specificity of CT scanning Thus, any one of the markers having a specificity greater than 65% can be used to advantage as a diagnostic assay for lung cancer as the instant assay would be as efficient in diagnosing lung cancer as the current standard, and delivered at lower cost and in a more non-invasive manner.

Also, it is noted that the five markers together provide greater predictive power, whatever the metric, than any one marker. The markers may be predictive in different subpopulations or the expression of two or more of the markers may be coordinated, for example, they may share a common biological presence or function. The aggregate predictive value is not necessarily additive and different combinations of the markers can provide different degrees of predictive accuracy. The statistic treatment used maximized predictive power and the five marker combination was the result based on the reference populations studied. Thus, a patient sample is tested with the five markers and the diagnosis, in principle, is calculated based on the five markers, because of the coordinated presence of two or more of the markers and the diagnostic metric based on the plurality of markers, such as one of the five marker panels taught hereinbelow. As discussed herein, because of the statistic treatment, such as logistic regression, any one of the variables contributing to the multivariable metric may have a greater or lesser contribution to the maximized total. If a patient has a score, a sum and the like that is at least 30%, at least 40%, at least 50%, at least 60% or greater of the aggregated metric of the five markers, even in circumstances where a patient may be negative for one or more of the markers, because of being positive some or more of the heavily weighted markers, that patient is considered more likely to be positive for lung cancer. The threshold score, sum and the like, which may be a reference or standard value, which may be a population mean value, and the acceptable level of patient/experimental sample similarity to that score, sum and the like to yield a positive test result, indicative of the possibility of the presence of lung cancer, is a design choice and may be determined by a statistical analysis that provides a confidence limit or level of detecting a positive sample or may be developed empirically, at the risk of a false positive. As taught hereinabove, that level can be at least 30%, at least 40%, at least 50%, at least 60% or greater, of the aggregated metric of the five markers or the population sum, the reference value and so on. The threshold or “tolerance”, that is, the degree of acceptable similarity of the patient score, sum and the like from the population score, sum and the like can be increased, that is, the patient score must be very near the population score, to increase sensitivity.

The predictive power of a marker or a panel can be measured using any of a variety of statistics, such as, specificity, sensitivity, positive predictive value, negative predictive value, diagnostic accuracy, AUC, of, for example, ROC curves which are a relationship between specificity and sensitivity, although it is known that the shape of the ROC curve is a relevant consideration of the predictive value, and so on, as known in the art.

The use of multiple markers enables a diagnostic test which is more robust and is more likely to be diagnostic in a greater population because of the greater aggregate predictive power of the plurality of markers considered together as compared to use of any one marker alone.

As discussed in greater detail hereinbelow, the instant invention contemplates the use of different assay formats. Microarrays enable simultaneous testing of multiple samples. Thus, a number of control samples, positive and negative, can be included in the microarray. The assay then can be run with simultaneous treatment of plural samples, such as a sample from one or more known affected patient samples, and one or more samples from normals, along with one or more samples to be tested and compared, the experimentals, the patient sample, the sample to be tested and so on. Including internal controls in the assay allows for normalization, calibration and standardization of signal strength within the assay. For example, each of the positive controls, negative controls and experimentals can be run in plural, and the plural samples can be a serial dilution. The control and experimental sites also can be randomly arranged on the microarray device to minimize variation due to sample site location on the testing device.

Thus, such a microarray or chip with internal controls enables diagnosis of experimentals (patients) tested simultaneously on the microarray or chip. Such a multiplex method of testing and data acquisition in a controlled manner enables the diagnosis of patients within an assay device as the suitable controls are accounted for and if the panel of markers are those which individually have a reasonably high predictive power, such as, for example, an AUC for an ROC curve of >0.85, and a total AUC across the five markers of >0.95, then a point of care diagnostic result can be obtained.

The assay can be operated in a qualitative way when each of the markers of a panel is found to have relatively comparable characteristics, such as those of the examples below. Thus, a lung cancer patient sample likely will be positive for all five markers, and such a sample, is very likely to be lung cancer positive. That would be validated by determining the odds based on the five markers as a whole as discussed herein, obtaining the sum or score of a metric of the five markers for the patient and then comparing that figure to the predictive power of the markers, derived using a statistical tool as discussed hereinabove. A patient positive for four of the markers, because the power of the four markers likely remains substantial, also should be considered at risk, could be diagnosed with lung cancer and/or should be examined in greater detail. A patient positive for only three markers might trigger a need for a retest, a test using other markers, a radiographic or other test, or may be called for another testing with the instant assay within another given interval of time.

Hence, for a panel of n markers, there is a derived predictive power formula, such as a regression formula, that defines the maximal likelihood graph defining the relationship of the five markers to the outcome. The patient may be positive for less than n markers in which case the patient may be considered positive or likely to be positive for further consideration when a majority, say 50% or more than half, of the markers are present in that patient. Also, should the patient present with overt signs potentially symptomatic of a lung disorder, as some panels may be specific for a particular disease, such as NSCLC, it may be that the patient needs to be further analyzed to rule out other lung disorders.

Thus, in any one assay using n markers, a preliminary, qualitative result can be obtained based on the gross number of positive signals of the total number of markers tested. A reasonable threshold may be to be positive for 50% or more of the markers. Thus, if four markers are tested, a sample positive for 2, 3 or 4 of the markers may be presumptively considered as possibly having lung cancer. If five markers are tested, a sample positive for 3, 4 or 5 markers may be considered presumptively positive. The threshold can be varied as a design choice.

Based on the acquisition and statistical treatment of data, from the standpoint of a population, an optimized panel of markers may be dynamic and may vary over time, may vary with the development of new markers, may vary as the population changes, increases and so on.

Also, as the tested population increases in size, the confidence of the marker subset, weighted coefficients and the likelihood of accurate probability of diagnosis may become more certain if the markers are biological or mechanistically related, and thus deviations, confidence limits or error limits will decrease. Therefore, the invention also contemplates use of a subset of markers which are usable in the general population. Alternatively, an assay device of interest may contain only a subset of markers, such as the panel of five markers that were used in the examples taught hereinbelow, which are optimized for a certain population.

Phage clone inserts encoding polypeptides can be analyzed to determine the amino acid sequence of the expressed polypeptide. For example, the phage inserts can be PCR-amplified using commercially available phage vector primers. Unique clones are identified based on differences in size and enzyme digestion pattern of the PCR products and the unique PCR products then are purified and sequenced. The encoded polypeptides are identified by comparison to known sequences, such as, the GenBank database using the BLAST search program.

Thus, for example, Tables 1 and 2 below summarize T7 phage clones of lung cancer cDNA which bind autoantibody in lung cancer patients.

TABLE 1

| Phage | ID - Gene | |
|---|---|---|
| Clone # | Symbol | Peptide Sequence |
| PC84* | ZNF440 | TLERNHVNVNSVVNPLVILLPIEYIK |
| | | ELTLEKSLMNIRNVGKHFIVPDPIVD |
| | | MKGFTWEKRLINVRNVEKHSRVPV |
| | | MFVYMKGPTLGKISMNVSSVGKHY |
| | | PLLQVFKHT (SEQ ID NO: 1) |
| PC87 | STK2 | GKVDVTSTQKEAENQRRVVTGSV |
| | | SSSRSSEMSSSKDRPLSARERRR |
| | | QACGRTRVTS (SEQ ID NO: 2) |
| PC125 | SOCS5 | SRRNQNCATEIPQIVEISIEKDNDS |
| | | CVTPGTRLARRDSYSRHAPWGGK |
| | | KKHSCSTKTQSSLDADKKF |
| | | (SEQ ID NO: 3) |
| PC123 | RPL4 | RNTILQARNHKLRVDKAAAAAAAA |
| | | LQAKSDEKAAVAGKKPVVGKKGK |
| | | ACGRTRVTS (SEQ ID NO: 4) |
| PC88 PC114 | RPL15 | YWVGEDSTYKFFEVILIDPFHKAIR |
| PC12688 | | RNPDTQWITKPVHKHREMRGLTS |
| | | AGRKSRGLGKGHKFHHTIGGSRR |
| | | AAWRRRNTLQLHRYR (SEQ ID |
| | | NO: 5) |
| PC40 | NPM1 | KLLSISGKRSAPGGGSKVPQKKVK |
| | | LAADEDDDDDDEEDDDEDDDDDD |
| | | FDDEEAEEKAPVKKSIRDTPAKN |
| | | (SEQ ID NO: 6) |
| PC20 PC22 | p130 | NKPAVTTKSPAVKPAAAPKQPVGG |
| G1802 | | GQKLLTRKADSSSSEEESSSSEEE |
| | | KTKKMVATTKPKATAKAALSLPAK |
| | | QAPQGSRDSSSDSDSSSSEEEEE |
| | | KTSKSAVKKKPQKVAGGAAPXKPA |
| | | SAKKGKAESSNSSSSDDSSEEE |
| | | (SEQ ID NO: 7) |
| PC57 | NFI-B | ASFPQHHHPGIPGVAHSVISTRTPP |
| | | PPSPLPFPTQAILPPAPSSYFSHPTI |
| | | RYPPHLNPQDTLKNYVPSYDPSSP |
| | | QTSQSWYLG (SEQ ID NO: 8) |
| PC94 | HMG14 | PKRRSARLSAKPPAKVEAKPKKAA |
| | | AKDKKSSDKKVQTKGKRGAKGKQA |
| | | EVANQETKEDLPAENGETKTEESP |
| | | ASDEAGEKEAKSD (SEQ ID |
| | | NO: 9) |
| PC16 | COX4 | AMFFIGFTALVIMWQKHYVYGPLP |
| | | QSFDKEWVAKQTKRMLDMKVNPI |
| | | QGLASKWDYEKNEWKK |
| PC112 | SFRS11 | ATKKKSKDKEKDRERKSESDKDVK |
| | | VTRDYDEEEQGYDSEKEKKEEKK |
| | | PIETGSPKTKECSVEKGTGDS |
| | | (SEQ ID NO: 11) |
| PC91 | AKAP12 | ESFKRLVTPRKKSKSKLEEKSEDSI |
| | | AGSGVEHSTPDTEPGKEESWVSIK |
| | | KFIPGRRKKRPDGKQEQAPVEDA |
| | | GPTGANEDDSDVPAVVPLSEYDAV |
| | | EREKLAAALE (SEQ ID NO: 12) |
| L1864 L1873 | GAGE7 | 5'3' Frame 1 |
| L1862 L1804 | | MLGDPNSSRPSSSVMKWNQQHLK |
| | | KGNQQLNVRILQLLRRERMREHLQ |
| | | VKGRSLKLIVRNRVTHRLGVSVKM |
| | | VLMGRRWTRQIQRR (SEQ ID |
| | | NO: 13 |
| | | 5'3' Frame 3 |
| | | ARGSEFKSPEQFSDEVEPATPEEG |
| | | EPATQRQDPAAAQEGEDEGASAG |
| | | QGPKPEAHSQEQGHPQTGCECED |
| | | GPDGQEMDPPNPEEVKTPEEGEK |
| | | QSQC (SEQ ID NO: 14) |
| G922 | Plakophillin | Frame 3 |
| | | ARGSEFKHGTVELQGSQTALYRT |
| | | GSVGIGNLQRTSSQRDTLTYQRNN |
| | | YALNTTATYAEPYRPIQYRVQECN |
| | | YNRLQHAVPADDGTTRSPSIDSIQ |
| | | DHARQTPWGPSEACGRTRVTS |
| | | (SEQ ID NO: 15) |
| L1747 | EEFIA | 5'3' Frame 3 |
| | | LAFVPISGWNGDNMLEPSANMPW |
| | | FKGWKVTRKDGNASGTTLLEALDC |
| | | ILPPTRPTDKPLRLPLQDVYKIGGIG |
| | | TVPVGRVETGVLKPGMVVTFAPVN |
| | | VTTEVKSVEMHHEA (SEQ ID |
| | | NO: 16) |
| L1761 | PMS2L15 | 5'3' Frame 1 |
| | | MLGDPNSSISLKFQAMDVG (SEQ |
| | | ID NO: 17) |
| | | 5'3' Frame 3 |
| | | ARGSEFKHLIEVSGNGCGEEENF |
| | | EGLISFSSETSHI (SEQ ID |
| | | NO: 18) |
| G2004 G313 | Paxillin | |
| G1896 G1750 | (PXN) | |
| L1857 L1839 | | |
| G1792 G1923 | | |
| PC6 PC8 | RAB7 | |

*The alphabet portion of the phage clone name in this and succeeding tables is fixed as a laboratory designation. As used herein, the numerical portion of the phage clone name is unambiguous identification of a clone.
**Redundant clones.

Table 2 provides other clones identified as associated with NSCLC that do not appear to encode a known polypeptide.

TABLE 2

| Phage | ID - Gene | |
|---|---|---|
| Clone # | Symbol | Nucleotide Sequence |
| L1896 | BAC clone | TCCGGGGACGAATTCCTGGTAGC |
| | RP11-499F19 | CTCATTCAGCCGATGGAAGGTAG |
| | | AAGGGACTCAGAACTTCAGGCCT |
| | | NATTCTGCGTTTTTGTATGCCCCA |
| | | AGAATGAAAGGGCTCTTTGTGAA |
| | | TTTGCATGTAGATTTATTTAACAT |
| | | TCAACCGGCAGAAAACGGAAGGT |
| | | AGTGCATGACACTGGGGGGAAC |
| | | CAGGCCCCCGCCCACCTCACATC |
| | | GTCATGGCATTAGCTGTTTACTG |
| | | GCTCCCGTGGAAACATTGGAAGG |
| | | GGATTTGTTTTGTGGTTGGGTTTC |
| | | CTTTTTTTTTTTTTTTTAACCAG |
| | | (SEQ ID NO: 24) |
| L1919 | SEC15L2 | GATTCTTCCTACCTTTGTCAGCTA |
| | | CTGAGTTGCTTCTGGGGAGGGAA |
| | | GTACTTCCTTGCCCCTCCCCAAC |
| | | CCCCCTACCTCACCATATCCTAT |
| | | CATATCTTGATAGTCATGGGGAA |
| | | GAGGATGTGCACACAGACATACA |
| | | AATTTCCTCAAAGCTGGAGAGAC |
| | | CAGGCTACATGTGAGCTCATAGA |
| | | TGCTGCTGAGGCTCATCCTGAGG |
| | | GCTGGATGGTTGGCCAGGGTTTC |
| | | AGAATGAGGGTAAGGGATGAGCA |
| | | CTGCCACCCAAGCTTGCGGCCG |
| | | CACTCGAGTAACTAGTTAACCCC |
| | | TTGGGGCCTCTAAACGGGTCTTG |
| | | AGGGGTTAANTAGTGACTCGAGT |
| | | GCGGCCGCA (SEQ ID NO: 25 |
| L1761 | PMS2L15 | ATGCTCGGGGATCCGAATTCAAG |
| | | CATCTCATTGAAGTTTCAGGCAAT |
| | | GGATGTGGGGTAGAAGAAGAAAA |
| | | CTTCGAAGGCTTAATCTCTTTCAG |
| | | CTCTGAAACATCACACATCTAAGA |
| | | TTCGAGAGTTTGCCGACCTAACT |
| | | CGGGTTGAAACTTTTGGCTTTCA |
| | | GGGGAAAGCTCTGAGCTCACTTT |
| | | GTGCACTGAGTGATGTCACCATT |
| | | TCTACCTGCCACGTATCGGCGAA |
| | | GGTTGGGACTCGACTGGTGTTTG |
| | | ATCACGATGGGAAAATCATCCAG |
| | | AAAACCCCCTACCCCCACCCCAG |
| | | AGGGACCACAGTCAGCGTGAAG |
| | | CAGTTATTTTCTACGCTACCTGTG |
| | | CGCCATAAGGAATTTCAAAGGAA |
| | | TATTAAGAAGTACAGAACCTGCTA |
| | | AGGCCATCAAACCTATTGATCGG |
| | | AAGTCAGTCCATCANATTTGCTCT |
| | | GGGCCGGTGGTACTGAGTCTAA |
| | | GCACTGCGGTGAAGAAGATAGTA |
| | | GGAAACAGTCTGGATGCTGGTGC |
| | | CACTAATATTGATCTAAAGCTTG |
| | | (SEQ ID NO: 26) |
| L1747 | EEFIA | GGGACGATTAGCTAGCATTTGTG |
| | | CCAATTTCTGGTTGGAATGGTGA |
| | | CAACATGCTGGAGCCAAGTGCTA |
| | | ACATGCCTTGGTTCAAGGGATGG |
| | | AAAGTCACCCGTAAGGATGGCAA |
| | | TGCCAGTGGAACCACGCTGCTTG |
| | | AGGCTCTGGACTGCATCCTACCA |
| | | CCAACTCGTCCAACTGACAAGCC |
| | | CTTGCGCCTGCCTCTCCAGGATG |
| | | TCTACAAAATTGGTGGTATTGGTA |
| | | CTGTTCCTGTTGGCCGAGTGGAG |
| | | ACTGGTGTTCTCAAACCCGGTAT |
| | | GGTGGTCACCTTTGCTCCAGTCA |
| | | ACGTTACAACGGAAGTAAAATCT |
| | | GTCGAAATGCACCATGAAGCTTG |
| | | CGGCCGCACTCGAGTAACTAGTT |
| | | AACCCCTTGGGGCCTCTAAACGG |
| | | GTCTTGGAGGGGTTAACNAGTTG |
| | | CTCGAGTGGGGCGGCNGGCTNC |
| | | TTGGTGGTTTATTTCAGA (SEQ |
| | | ID NO: 27) |
| G1954 | MALAT1 | CTCGGGGATCCGAATTTCAAGCG |
| | | GCAAGAAGTTTCAGAATAAGAAA |
| | | ATGAAAAACAAGCTAAGACAAGT |
| | | ATTGGAGAAGTATAGAAGATAGA |
| | | AAAATATAAAGCCAAAAATTGGAT |
| | | AAAATAGCACTGAAAAAATGAGG |
| | | AAATTATTGGTAACCAATTTATTTT |
| | | AAAAGCCCATCAATTTAATTTCTG |
| | | GTGGTGCAGAAGTTAGAAGGTAA |
| | | AGCTTGAGAAGATGAGGGTGTTT |
| | | ACGTAGACCAGAACCAATTTAGA |
| | | AGAATACTTGAAGCTAGAAGGGG |
| | | AAGCTTGCGGCCGCACTCGAGTA |
| | | ACTAGTTAACCCCTTGGGGCCTC |
| | | TAAACGGGTCTTGAGGGGTTAAC |
| | | TCGAGTTACTCGTGGGCGCAGCT |
| | | CTTTGCTTAGTATTTTTAATGGTT |
| | | GGTTGTAACCTTTCGTTTCTCATC |
| | | GCCGAATTATGATGGTTTTAAATA |
| | | ATGATCATAATTCTTTCTTTTTACT |
| | | TGGTTTTTTTTTTTCACTTTTACTT |
| | | TCTGTTTATGAAGCACGCCCGCC |
| | | CCACAA (SEQ ID NO: 28) |
| G1689 | XRCC5 | ATGCTCGGGGATCCGAATTCAGC |
| | | TTGGGAACGCGGCCATTTCAAAG |
| | | GGGAAGCCAAAATCTCAAGAAAT |
| | | TCCCAGCAGGTTACCTGGAGGC |
| | | GGATCATCTAATTCTCTGTGGAAT |
| | | GAATACACACATATATATTACAAG |
| | | GGATAAGCTTGCGGCCGCACTC |
| | | GAGTAACTAGTTAACCCCTTGGG |
| | | GCCTCTAAACGGGACTTGAGGG |
| | | GTAAGCTAGTTACTCGAGGGCGA |
| | | GCTTATGGGAAATATATATTGCG |
| | | GTATTTAAGGAATTAGTTACCCGC |
| | | TCGCTGGCCTTTGAACTGTTGTTT |
| | | GAGGCCTTAAATTGATGATCGTG |
| | | GTGGGAAACAAGAGGTGGGGTG |
| | | GGAGATTTGTTTTTTGTTCTGAAG |
| | | CGGGGAGGGGACTAGACCCTAA |
| | | AAGCATTTAAATATAAGACAACCC |
| | | AAT (SEQ ID NO: 29) |
| L1829 | BMI-1 | GGTACGAATTAGCCAGANATCGG |
| L1841 | | GGCGAGTACAATGGGGATGTGG |
| L1676 | | GCGCGGGAGCCCCGCTCCCCTT |
| L1916 | | TTTTAGCAGCACCTCCCAGCCCC |
| | | GCAGAATAAAACCGATCGCNNCC |
| | | CCTCCGCGCGCGCCCTCCCCCG |
| | | AGATGCGGAGCGGGAGGAGGCG |
| | | GCGGCGGCCGAGGAGGAGGAG |
| | | GAGGAGGCCCCGGAGGAGGAGG |
| | | CGTTGGAGGTCGAGGCGGAGGC |
| | | GGAGGAGGAGGAGGCCGAGGC |
| | | GCCGGANGAGGCCNAGGCGCCG |
| | | GAGCAGGAGGAGGCCGGCCGGA |
| | | GGCGGCATGAGACGAGCGTGGC |
| | | GGCCGCGGCTGCTCGGGGCCGC |
| | | GCTGGTTGCCCATTGACAGCGGC |
| | | GTCTGCAGCTCGCTTCAAGATGG |
| | | CCGCTTGGCTCGCATTCATTTTCT |
| | | GCTGAACGACTTTTAACTTTCNTT |
| | | GTCTTTTCCGCCCGCTTCNATCG |
| | | CCTCNCGCCGGCTGCTCTTTCCG |
| | | GGATTTTTTATCAAGCAGAAATGC |
| | | ATCG (SEQ ID NO: 31) |

Random peptide libraries also can be used to identify candidate polypeptides that bind circulating antibodies in NSCLC patients but not in normals. Thus, for example, a phage display peptide library comprising 10⁹ random peptides fused to a virus minor coat protein can be screened for capture proteins that bind lung cancer patient antibody using techniques similar to that described above, such as using microarrays, and as known in the art. One M 13 library that was used (New England Biolabs) expresses a 7 amino acid polypeptide insert as a loop structure on the phage surface.

As described herein, the library is biopanned to enrich for phage-expressed proteins that are specifically recognized by circulating antibodies in NSCLC patient serum. Phage lysates of selected clones are robotically spotted (Affymetrix, Santa Clara, Calif.) in duplicate on slides (Schleicher and Schuell, Keene, N.H.). The arrayed phage are incubated with a serum sample from a patient with NSCLC to identify phage-expressed proteins bound by circulating lung tumor-associated antibodies.

Using a known immunoassay, with suitable reporter molecules, computer generated regression lines that indicate the mean signal and standard deviation of all polypeptides on the slide, are used to identify peptides that were bound by antibody in NSCLC patient plasma. Phage binding significant amounts of antibody from an NSCLC plasma sample (for example, >3 standard deviations from the norm) are considered candidates for further evaluation.

TABLE 3
M13 Clones

| Phage ID | Nucleotide Sequence | Amino Acid Sequence (3 letter) |
|---|---|---|
| MC0457 | ATTGTGAATAAGCATAAGGTT | Ile Val Asn Lys His Lys Val |
| | (SEQ ID NO: 32) | |
| MC0908 | GAGCGGTCTCTGAGTCCGATT | Glu Arg Ser Leu Ser Pro Ile |
| | (SEQ ID NO: 33) | |
| MC0919 | TTGAGTCAGAATCCGCATAAG | Leu Ser Gln Asn Pro His Lys |
| | (SEQ ID NO: 34) | |
| MC1484 | AATGCGAGTCATAAGTGTTCT | Asn Ala Ser His Lys Cys Ser |
| | (SEQ ID NO: 35) | |
| MC1509 | AATGCGCTGGCTAATCCTTCG | Asn Ala Leu Ala Asn Pro Ser |
| | (SEQ ID NO: 35) | |
| MC1521 | GCGAAGCCGCCGAAGCTGTCT | Ala Lys Pro Pro Lys Leu Ser |
| | (SEQ ID NO: 37) | |
| MC1524 | AGGGCTCTGGATCCGGATTCG | Arg Ala Leu Asp Pro Asp Ser |
| | (SEQ ID NO: 38) | |
| MC1760 | ATACTACTGGGTCGCCTCTGT | Ile Leu Leu Gly Arg Leu Cys |
| | (SEQ ID NO: 39) | |
| MC1786 | AAGGTTAATACTCATCATACT | Lys Val Asn Thr His His Thr |
| | (SEQ ID NO: 40) | |
| MC2541 | CTGTTTCTGACGGCGCAGGCG | Leu Phe Leu Thr Ala Gln Ala |
| | (SEQ ID NO: 41) | |
| MC2720 | TTTAATTGGTATAATTCGTCG | Phe Asn Trp Tyr Asn Ser Ser |
| | (SEQ ID NO: 42) | |
| MC2729 | CTTCCGCATCAGCTGCGGTGG | Leu Pro His Gln Leu Ala Trp |
| | (SEQ ID NO: 43) | |
| MC2853 | CTTGCGTGGTATGCGAAGAGT | Leu Ala Trp Tyr Ala Lys Ser |
| | (SEQ ID NO: 44) | |
| MC2900 | AAGATTGGGACGGCGTGGCTT | Lys Ile Gly Thr Ala Trp Leu |
| | (SEQ ID NO: 45) | |
| MC2986 | ACGCCTACTCATGGTGGGAAG | Thr Pro Thr His Gly Gly Lys |
| | (SEQ ID NO: 46) | |
| MC2996 | ACTCCTACTTATGCGGGGTAT | Thr Pro Thr Tyr Ala Gly Tyr |
| | (SEQ ID NO: 47) | |
| MC2998 | ATGCCGGCTACTACGCCTCAG | Mat Pro Ala Thr Thr Pro Gln |
| | (SEQ ID NO: 48) | |
| MC3000 | AAGGCGTGGTTTGGGCAGATT | Lys Ala Trp Phe Gly Gln Ile |
| | (SEQ ID NO: 49) | |
| MC3018 | AAGAATTGGTTTGGTCATACG | Lys Asn Trp Phe Gly His Thr |
| | (SEQ ID NO: 50) | |
| MC3023 | CATACTCATCATGATAAGCAT | His Thr His His Asp Lys His |
| | (SEQ ID NO: 51) | |
| MC3046 | ATTACGAATAAGTGGGGGTAT | Ile Thr Asn Lys Trp Gly Tyr |
| | (SEQ ID NO: 52) | |
| MC3050 | CTGAATACGCATTCGTCTCAG | Leu Asn Thr His Ser Ser Gln |
| | (SEQ ID NO: 53) | |
| MC3143 | GGGCCTGCGTGGGAGGATCCG | Gly Pro Ala Trp Glu Asp Pro |
| | (SEQ ID NO: 54) | |
| MC3146 | AGTCAGTCTTATCATAAGCGTAC | Ser Gln Ser Tyr His Lys Arg |
| | TAGC (SEQ ID NO: 55) | Thr Ser |

Additional lung cancer-specific clones not yet sequenced are provided in Table 4 below.

TABLE 4
M13 Clones
Phage ID

MC1011
MC2106
MC2628
MC2829
MC3052
MC3096
MC1805
MC2238
MC2645
MC3047
MC3156
MC3090
MC2987
MC3019
MC3045
MC3048
MC3135

The objective of the high throughput screening of libraries is not to identify all cancer-specific proteins, but rather to identify a cohort of predictive markers that as a panel can be used to predict the inclusion of a subject into a lung cancer cohort or not with a maximal degree of specificity and sensitivity. As such, the approach is not targeted to generating a comprehensive proteomic profile, or to identify per se, disease proteins, such as lung cancer proteins, but to identify a number of markers that are predictive of disease and when aggregated as a panel, enable a robust predictive assay for a heterogeneous disease in a heterogeneous population. Any one marker may or may not have a direct role in lung oncogenesis, or as a peptide, the actual role of the molecule from which the peptide originates may be unknown at the present.

### Measuring Antibody Binding to Individual Capture Proteins

Capture proteins compiled on a diagnostic chip can be used to measure the relative amount of lung cancer-specific antibodies in a blood sample. This can be accomplished using a variety of platforms, different formulations of the polypeptide (e.g. phage expressed, cDNA derived, peptide library or purified protein), and different statistical permutations that allow comparison between and among samples. Comparison will require that measurements be standardized, either by external calibration or internal normalization. Thus, in the exemplified glass slide array comprised of multiple phage-expressed capture proteins (for example, M 13 and T7 phage) and multiple negative external control proteins (phages not bound by antibodies in patient plasmas and M1 3 or T7 phages that have no inserts—called “empty” phages) using an immunoassay as the screening means, the data were normalized by two color fluorescent labeling of phage capsids and plasma sample antibody binding using two non-limiting statistical approaches:

1) Antibody/Phage Capsid Signal Ratio

Capture proteins identified in screening, multiple nonreactive phages, plus “empty” phages on single diagnostic chips are incubated with sample(s) using standard immunochemical techniques and dual color staining. The median (or mean) signal of antibody binding the capture protein is divided by the median (or mean) signal of a commercial antibody against phage capsid protein to account for the amount of total protein in the spot. Thus, the plasma/phage capsid signal ratio (for example, Cy5/Cy3 signal ratio) provides a normalized measurement of human antibody against a unique phage-expressed protein.

Measurements then can be further normalized by subtracting background reactivity against empty phage and dividing by the median (or mean) of the phage signal, [(Cy5/Cy3 ofphage)-(Cy5/Cy3 of empty phage)/(Cy5/Cy3 of empty phage)]. This methodology is quantitative, reproducible, and compensates for chip-to-chip variability, allowing comparison of samples.

2) Standardized Residual

Capture proteins identified in screening, multiple nonreactive phages, plus “empty” phages on single diagnostic chips are incubated with sample(s) using standard immunochemical techniques and dual color staining. The distance from a statistically determined regression line is measured, then standardized by dividing that measure by the residual standard deviation. This approach also affords a reliable measure of the amount of antibody binding to each unique phage-expressed protein over the amount of protein in each spot, is quantitative, reproducible, and compensates for chip-to-chip variability, allowing comparison of samples.

Such a normalization of signal can be used with the unknowns being tested in a diagnostic assay to determine whether a patient is positive or not for a marker. The assay can rely on a qualitative determination of antibody presence, for example, any normalized value above background is considered as evidence of that antibody. Alternatively, the assay can be quantified by determining the strength of the signal for a marker, as a reflection of the vigor of the antibody response. Thus, the actual numerical normalized value of a reaction to a marker can be used in the formulaic determination of diagnosing cancer as described Herein.

Identifying predictive markers

Normalized measurements of all candidate phage-expressed proteins can be independently analyzed for statistically significant differences between a patient group and normal group, for example, by t-test using JMP statistical software (SAS, Inc., Cary, N.C.). Various combinations of markers with differing levels of independent discrimination for samples tested can be statistically combined in a variety of ways. The statistical treatment is one which compares, in a multivariable analytical fashion, all of the markers in various combinations to obtain a panel of markers with maximal likelihood of being associated with the presence of disease. As in any population statistic, the selection of markers is dictated by the number and type of samples used. As such, an “optimal combination of markers” may vary from population to population or be based on the stage of the anomaly, for example. An optimal combination of markers may be altered when tested in a large sample set (>1000) based on variability that may not be apparent in smaller sample sizes (<100) or may demonstrate reduced deviation because of validation of population prevalence of the marker. Weighted logistic regression is a logical approach to combining markers with greater and lesser independent predictive value. An optimal combination of markers for discriminating the samples tested can be defined by organizing and analyzing the data using ROC curves, for example.

### Class Prediction

Standardized responses for all candidate phage-expressed proteins are independently analyzed for statistically significant differences between a patient group and a normal group, for example, by t-test. The statistical treatment is one which compares, in a multivariable analytical fashion, all of the markers in various combinations to obtain a panel of markers with maximal likelihood of being associated with the presence of cancer.

The panels (combined measures of two or more markers) exemplified herein for lung cancer have a high combined predictive value and demonstrate excellent discrimination (cancer yes vs. cancer no). While the present invention includes particular peptide panels which were chosen for the ability to discriminate between available cancer and normal samples, it will be appreciated that the invention has been developed using some, but not all identified markers, and not all potentially identifiable markers, or combinations thereof. Thus, a panel may comprise at least two markers; at least three markers; at least four markers; at least five markers; at least six markers; at least seven markers; at least eight markers; at least nine markers; at least ten markers and so on, the number of markers governed by the statistical analysis to obtain maximal predictability of outcomes. Thus, for example, the examples and panels described herein are examples only.

From a statistical standpoint, inclusion of additional markers ultimately will lead to a test which will identify all affected individuals in a sample. However, a commercial embodiment may not require or need or want a large number of markers because of cost considerations, the statistical treatments that may be required because a larger number of variables are being considered, perhaps the need for a greater number of controls thereby reducing the number of experimentals that can be tested at one time and so on. Commerciability has different endpoints from scientific certainty.

However, the observation that a greater number of markers or a different panel of markers can enhance sensitivity and/or specificity leads to the embodiment where follow up studies subsequent to a positive assay with a small number of markers will have the patient sample tested with a smaller or larger number of markers, or a different panel of markers to rule out the possibility of a false positive. Such follow up studies using an assay of interest with a reconfigured panel of biomarkers is an attractive alternative to more costly and potentially invasive techniques, such as CT which exposes the patient to high levels of radiation, or a biopsy. Thus, for example, a patient that is positive for three or less of a five-marker panel, may be tested with a larger panel of markers as a confirmatory test.

The instant assay also can serve as confirmation of another assay format, such as an X-ray or CT scan, particularly if the X-ray or CT scan is one which does not provide a definitive diagnosis, which would lead to the need for retesting, for a quick follow-up, a protracted or shortened period until the next test and so on. Thus, an instant assay can be used as a follow-up in such patients. A positive test would confirm the likelihood of lung cancer, and a negative test would indicate either a benign cancer or no cancer at all, and the non-diagnostic X-ray or CT scan revealed a normal tissue variation.

Since accurate class prediction in a “commercial ready” assay will be based on measurements from a large number of samples from a broad demographic, all retrospective sample testing during development can ultimately be incorporated as classifiers, and the power of the assay, such as the predictive value, will be continually improved. In addition to this dynamic aspect of assay development, the nature of a multiplex (multi marker) assay allows predictive markers to be added at any point in development or implementation.

In context, validating markers for use in diagnosis will serve the secondary purpose of generating a highly stable set of classifiers that enhance the predictive accuracy by defining a “normal range”. Deviation from that normal range will provide a statistical probability of disease (for example >2 standard deviations from the norm) although cutoff values that are most appropriate for clinical diagnostics will have to be determined by the variability in a given target population.

### Multiple Marker Assays and Application

As discussed in greater detail herein, the instant invention contemplates the use of different assay formats. Microarrays enable simultaneous testing of multiple samples. Thus, a number of control samples, positive and negative, can be included in the microarray. Hence, the assay can be run with simultaneous treatment of plural samples, such as a sample from a known affected patient and a sample with a normal, along with a sample to be tested. Running internal controls allows for normalization, calibration and standardization of signal strength within the assay.

Thus, such a microarray, MEMS device, NEMS device or chip with internal controls enables point of care diagnosis of experimentals (patients) tested simultaneously on the device. The MEMS and NEMS devices can be ones used for the microarray assays, or can be in a “lab on a chip” format, such as incorporating microfluidics and so on which would enable additional assay formats and reporters.

To enhance predictive power and value, and applicability across general populations, and to reduce costs, the instant assay format can range from standard immunoassays, such as dipstick and lateral flow immunoassays, which generally detect one or a small number of targets simultaneously at low manufacturing cost, to ELIS A-type formats which often are configured to operate in a multiple well culture dish which can process, for example, 96, 384 or more samples simultaneously and are common to clinical laboratory settings and are amenable to automation, to array and microarray formats where many more samples are tested simultaneously in a high throughput fashion. The assay also can be configured to yield a simple, qualitative discrimination (cancer yes vs. cancer no).

But multiple different applications in disease management are possible and markers unique for any one application can be made as taught herein. Different sets of markers are obtained for distinguishing lung cancer from other types of cancer, distinguishing early from late stage cancer, distinguishing specific subtypes of cancer and for following the progression of disease after therapeutic intervention. Thus, a treatment regimen can be assessed and manipulated as needed by repeated serial testing with the instant assay to monitor the progress of treatment or remission. A quantitative version of the assay, for example, by containing a serial dilution of capture molecules, can discriminate diminution of cancer size with treatment.

Once the particular epitopes, such as peptides are identified for detecting circulating autoantibody, the particular epitopes can be used in diagnostic assays, in formats known in the art. As the interaction is an immune reaction, a suitable diagnostic can be presented in any of a variety of known immunoassay formats. Thus, an epitope can be affixed to a solid phase, for example, using known chemistries. Also, the epitopes can be conjugated to another molecule, often larger than the epitope to form a synthetic conjugate molecule or can be made as a composite molecule using recombinant methods, as known in the art. Many polypeptides naturally bind to plastic surfaces, such as polyethylene surfaces, which can be found in tissue culture devices, such as multiwell plates. Often, such plastic surfaces are treated to enhance binding of biologically compatible molecules thereto. Thus, the polypeptides form a capture element, a liquid suspected of carrying an autoantibody that specifically binds that epitope is exposed to the capture element, antibody becomes affixed and immobilized to the capture element, and then following a wash, bound antibody is detected using a suitable detectably labeled reporter molecule, such as an anti-human antibody labeled with a colloidal metal, such as colloidal gold, a fluorochome, such as fluorescein, and so on. That mechanism is represented, for example, by an ELISA, RIA, Western blot and so on. The particular format of the immunoassay for detecting autoantibody is a design choice.

Alternatively, as particular phage express an epitope specifically bound by autoantibodies found in patients with lung cancer (which clones are specifically named and stored as stocks, and will be made available on request when a patent matures from the instant application), the capture element of an assay can be the individual phage, such as obtained from a cell lysate, each at a capture site on a solid phase. Also, a reactively inert carrier, such as a protein, such as albumin and keyhole limpet hemocyanin, or a synthetic carrier, such as a synthetic polymer, to which the expressed epitope is attached, similar to a hapten on a carrier, or any other means to present an epitope of interest on the solid phase for an immunoassay, can be used.

Alternatively, a format may take the configuration wherein a capture element affixed to a solid phase is one which binds to the non-antigen-binding portions of immunoglobulin, such as the F_{c} portion of antibody. Accordingly, a suitable capture element may be Protein A, Protein G or and a-F_{c} antibody. Patient plasma is exposed to the capture reagent and then presence of lung cancer-specific antibody is detected using, for example, labeled marker in a direct or competition format, as known in the art.

Similarly, the capture element can be an antibody which binds the phage displaying the epitope to provide another means to produce a specific capture reagent, as discussed above.

As known in the immunoassay art, the capture element is a determinant to which an antibody binds. As taught herein, the determinant may be any molecule, such as a biological molecule, or portion thereof, such as a polypeptide, polynucleotide, lipid, polysaccharide, and so on, and combinations thereof, such as glycoprotein or a lipoprotein, the presence of which correlates with presence of an antibody found in lung cancer patients. The determinant can be naturally occurring, and purified, for example. Alternatively, the determinant can be made by recombinant means or made synthetically, which may minimize cross reactivity. The determinant may have no apparent biological function or not necessarily be associated with a particular state, however, that does not detract from the use thereof in a diagnostic assay of interest.

The solid phase of an immunoassay can be any of those known in the art, and in forms as known in the art. Thus, the solid phase can be a plastic, such as polystyrene or polypropylene, a glass, a silica-based structure, such as a silicon chip, a membrane, such as nylon, a paper and so on. The solid phase can be presented in a number of different and known formats, such as in paper format, a bead, as part of a dipstick or lateral flow device, which generally employ membranes, a microtiter plate, a slide, a chip and so on. The solid phase can present as a rigid planar surface, as found in a glass slide or on a chip. Some automated detector devices have dedicated disposables associated with a means for reading the detectable signal, for example, a spectrophotometer, liquid scintillation counter, colorimeter, fluorometer and the like for detecting and reading a photon-based signal.

Other immune reagents for detecting the bound antibody are known in the art. For example, an anti-human Ig antibody would be suitable for forming a sandwich comprising the capture determinant, the autoantibody and the anti-human Ig antibody. The anti-human Ig antibody, the detector element, can be directly labeled with a reporter molecule, such as an enzyme, a colloidal metal, radionuclide, a dye and so on, or can itself be bound by a secondary molecule that serves the reporter function. Essentially, any means for detecting bound antibody can be used, and such any means can contain any means for a reporting function to yield a signal discernable by the operator. The labeling of molecules to form a reporter is known in the art.

In the context of a device that enables the simultaneous analysis of a multitude of samples, a number of control elements, both positive and negative controls can be included on the assay device to enable controlling for assay performance, reagent performance, specificity and sensitivity. Often, as mentioned, much, if not all of the steps in making the device of interest and many of the assay steps can be conducted by a mechanical means, such as a robot, to minimize technician error. Also, the data from such devices can be digitized by a scanning means, the digital information is communicated to a data storage means and the data also communicated to a data processing means, where the sort of statistical analysis discussed herein, or as known in the art, can be effected on the data to produce a measure of the result, which then can be compared to a reference standard or internally compared to present with an assay result by a data presentation means, such as a screen or read out of information, to provide diagnostic information.

For devices which analyze a smaller number of samples or where sufficient population data are available, a derived metric for what constitutes a positive result and a negative result, with appropriate error measurements, can be provided. In those cases, a single positive control and a single negative control may be all that is needed for internal validation, as known in the art. The assay device can be configured to yield a more qualitative result, either included or not in a lung cancer cluster, for example.

Other high throughput and/or automated immunoassay formats can be used as known and available in the art. Thus, for example, a bead-based assay, grounded, for example, on colorimetric, fluorescent or luminescent signals, can be used, such as the Luminex (Austin, Tex.) technology relying on dye-filled microspheres and the BD (Franklin Lakes, N.J.) Cytometric Bead Array system. In either case, the epitopes of interest are affixed to a bead.

Another multiplex assay is the layered arrays method of Gannot et al., J. Mol. Diagnostics 7, 427-436, 2005. The method relies on the use of multiple membranes, each carrying a different one of a binding pair, such as a target molecule, such as an antigen or a marker, the membranes configured in register to accept a sample which is suspected of carrying the other of the binding pair, for chromatographic transfer in register. The sample is allowed to wick or be transported through a number of aligned membranes to provide a three-dimensional matrix. Thus, for example, a number of membranes can be stacked atop a separating gel and the gel contents are allowed to exit the separating gel and pass through the stacked membranes. Any association of molecules between that affixed to any one membrane and that transported through the membrane stack, such as an antigen bound to an antibody, can be visualized using known reporter and detection materials and methods, see for example, U.S. Pat. Nos. 6,602,661 and 6,969,615; as well as U.S. Pub. Nos. 20050255473 and 20040081987.

In other embodiments, a composition or device of interest can be used to detect different classes of molecules associated or correlated with lung cancer. Thus, an assay may detect circulating autoantibody and non-antibody molecules associated or correlated with lung cancer, such as a lung cancer antigen, see, for example, Weynants et al., Eur. Respir. J., 10:1703-1719, 1997 and Hirsch et al., Eur. Respir. J., 19:1151-1158, 2002. Accordingly, a device can contain as capture elements, epitopes for autoantibodies and binding molecules for lung cancer molecules, such as specific antibodies, aptamers, ligands and so on.

### Exemplification of Sampling and Testing

Samples amenable to testing, particularly in screening assays, generally, are those easily obtainable from a patient, and perhaps, in a non-intrusive or minimally invasive manner. The sample also is one known to carry an autoantibody. A blood sample is a suitable such sample, and is readily amenable to most immunoassay formats.

In the context of a blood sample, there are many known blood collection tubes, many collect 5 or 10 ml of fluid. Similar to most commonly ordered diagnostic blood tests, 5 ml of blood is collected, but the instant assay operating as a microarray likely can require less than 1 ml of blood. The blood collection vessel can contain an anticoagulant, such as heparin, citrate or EDTA. The cellular elements are separated, generally by centrifugation, for example, at 1000×g (RCF) for 10 minutes at 4° C. (yielding −40% plasma for analysis) and can be stored, generally at refrigerator temperature or at 4° C. until use. Plasma samples preferably are assayed within 3 days of collection or stored frozen, for example at −2O⁰C. Excess sample is stored at −2O⁰C (in a frost-free refrigerator to avoid freeze thawing of the sample) for up to two weeks for repeated analysis as needed. Storage for periods longer than two weeks should be at −8O⁰C. Standard handling and storage methods to preserve antibody structure and function as known in the art are practiced.

The fluid samples are then applied to a testing composition, such as a microarray that contain sites loaded with, for example, samples of purified polypeptides of one of the five marker panels discussed herein, along with suitable positive and negative samples. The samples can be provided in graded amounts, such as a serial dilution, to enable quantification. The samples can be randomly sited on the microarray to address any positional effects. Following incubation, the microarray is washed and then exposed to a detector, such as an anti-human antibody that is labeled with a particular marker. To enable normalization of signal, a second detector can be added to the microarray to provide a measure of sample at each site, for example. That could be an antibody directed to another site on the isolated polypeptide samples, the polypeptide can be modified to contain additional sequences or a molecule that is inert to the specific reaction, or the polypeptides can be modified to carry a reporter prior to addition onto the microarray. The microarray again is washed, and then if needed, exposed to a reagent to enable detection of the reporter. Thus, if the reporter comprises colored particles, such as metal sols, no particular detection means is needed. If fluorescent molecules are used, the appropriate incident light is used. If enzymes are used, the microarray is exposed to suitable substrates. The microarray is then assessed for reaction product bound to the sites. While that can be a visual assessment, there are devices that will detect and, if needed, quantify strength of signal. That data then is interpreted to provide information on the validity of the reaction, for example, by observing the positive and negative control samples, and, if valid, the experimental samples are assessed. That information then is interpreted for presence of cancer. For example, if the patient is positive for three or more of the antibodies, the patient is diagnosed as positive for lung cancer. Alternatively, the information on the markers can be applied to the formula that describes the maximum likelihood relationship of the five markers together to the outcome, presence of lung cancer, and if the clue of a score of the patient is greater than 50% of the value of that same score of the panel, the patient is diagnosed as positive for cancer. A suitable score can be the calculated AUC values.

### Use of the Kit and Assay

The blood test according to the present invention has multiple uses and applications, although early diagnosis or early warning for subsequent follow up is highly compelling for its potential impact on disease outcomes. The invention may be employed as a tool to complement radiographic screening for lung cancer. Serial CT screening is generally sensitive for lung cancer, but tends to be quite expensive and nonspecific (64% reported specificity.) Thus, CT results in a high number of false positives, nearly four in ten. The routine identification of indeterminate pulmonary nodules during radiographic imaging frequently leads to expensive workup and potentially harmful intervention, including major surgery. Currently, age and smoking history are the only two risk factors that have been used as selection criteria by the large screening studies for lung cancer.

Use of the blood test according to the present invention to detect radiographically apparent cancers (>0.5 cm) and/or occult or pre-malignant cancer (below the limit of conventional radiographic detection) would define individuals for whom additional screening is most warranted. Thus, the instant assay can serve as the primary screening test, wherein a positive result is indication for further examination, as is conventional and known in the art, such as radiographic analysis, such as a CT, PET, X-ray and the like, hi addition, periodic retesting may identify emerging NSCLC.

An example of how the subject test may be incorporated into a medical practice would be where high risk smokers (for example, persons who smoked the equivalent of one pack per day for twenty or more years) may be given the subject blood test as part of a yearly physical. A negative result without any further overt symptoms could indicate further testing at least yearly. If the test result is positive, the patient would receive further testing, such as a repeat of the instant assay and/or a CT scan or X-ray to identify possible tumors. If no tumor is apparent on the CT scan or X-ray, perhaps the instant assay, would be repeated once or twice within the year, and multiple times in succeeding years until the tumor is at least 0.5 mm in diameter and can be detected and surgically removed.

As set forth in the Examples that follow, the −90% sensitivity of autoantibody profiling for NSCLC using an exemplified five-marker panels compares quite favorably to that of CT screening alone, and by comparison may perform especially well for small tumors, and represents an unparalleled advance in detection of occult disease. Moreover, the greater than 80% specificity of the instant assay well exceeds that of CT scanning, which becomes increasingly more important as the percentage of benign pulmonary nodules increases in the at-risk population, rising to levels of about 70% of participants in the Mayo Clinic Screening Trial, for example.

In addition to use in screening, the assay and method of the present invention may also be useful to the closely related clinical problem of distinguishing benign from malignant nodules identified on CT screening. The solitary pulmonary nodule (SPN) is defined as a single spherical lesion less than 3 cm in diameter that is completely surrounded by normal lung tissue. Although the reported prevalence of malignancy in SPNs has ranged from about 10% to about 70%, most recent studies using the modern definition of SPN reveal the prevalence of malignancy to be about 40% to about 60%. The majority of benign lesions are the result of granulomas while the majority of the malignant lesions are primary lung cancer. The initial diagnostic evaluation of an SPN is based on the assessment of risk factors for malignancy such as age, smoking history, prior history of malignancy and chest radiographic characteristics of the nodule such as size, calcification, border (spiculated, or smooth) and growth pattern based on the evaluation of old chest x-rays. These factors are then used to determine the likelihood of malignancy and to guide further patient management.

After an initial evaluation, many nodules will be classified as having an intermediate probability of malignancy (25-75%). Patients in this group may benefit from additional testing with the instant assay before proceeding to biopsy or surgery. Serial scanning assessing growth or metabolic imaging (e.g. PET scanning) are the only noninvasive options currently available and are far from ideal. Serial radiographic analysis relies on measures of growth, requiring a lesion show no growth over a two year timeframe; an ideal interval betweens scans has not been determined although CT scans every 3 months for two years is a conventional longitudinal evaluation. PET scan has 90-95% specificity for lung cancer and 80-85% sensitivity. These predictive values may vary based on regional prevalence of benign granulomatous disease (e.g. histoplasmosis).

PET scans currently cost between $2000 and $4000 per test. Diagnostic yields from non-surgical procedures such as bronchoscopy or transthoracic needle biopsy (TTNB) range from 40% to 95%. Subsequent management in the setting of a nondiagnostic procedure can be problematic. Surgical intervention is often pursued as the most viable option with or without other diagnostic workup. The choice will depend on whether the pretest risk of malignancy is high or low, the availability of testing at a particular institution, the nodule's characteristics (e.g., size and location), the patient's surgical risk, and the patient's preference. Previous history of other extrathoracic malignancy immediately suggests the possibility of metastatic cancer to the lung, and the relevance of noninvasive testing becomes negligible. In the confounding clinical scenario of SPN with indeterminate clinical suspicion for lung cancer, circulating tumor markers could help avoid potentially harmful invasive diagnostic workups and conversely support the rationale for aggressive surgical intervention.

The described invention thus enhances the clinical comfort of electing to serially image a nodule in lieu of invasive diagnostics. The invention also will have an influence in the interval for serial X-ray or CT screening, thereby lowering clinical health care costs. The described invention will complement or supplant PET scanning as a cost effective method to further increase the probability that lung cancer is present or absent.

The invention will be useful in assessing disease recurrence following therapeutic intervention. Blood tests for colon and prostate cancer are commonly employed in this capacity, where marker levels are followed as an indicator of treatment success or failure and where rising marker levels indicate the need for further diagnostic evaluation for recurrence that leads to therapeutic intervention.

The invention will provide important information about tumor characteristics; determining tumor subtypes with poor prognosis could significantly impact a clinical decision to recommend additional therapies with potential toxicity because the assay relies on multiple markers, any one of which may be characteristic of a particular cancer or a unique parameter thereof. Development of newer treatments used for long-term consolidation of conventional surgery or chemotherapy may require careful cost/benefit analysis and patient selection.

Hence, the instant assay will be a valuable tool for screening, choice of treatment and for continued use during treatment to monitor the course of treatment, success of treatment, relapse, cure and so on. The reagents of the instant assay, the particular panel of markers can be manipulated to suit the particular purpose. For example, in a screening assay, a larger panel of markers or a panel of very prevalent markers is used to maximize predictive power for a greater number of individuals.

However, in the context of an individual, undergoing treatment, for example, the particular antibody fingerprint of the patient tumor can be obtained, which may or may not require all of the markers used for screening, and that particularized subset of markers can be used to monitor the presence of the tumor in that patient, and subsequent therapeutic intervention.

The components of an assay of interest can be configured in a number of different formats for distribution and the like. Thus, the one or more epitopes can be aliquoted and stored in one or more vessels, such as glass vials, centrifuge tubes and the like. The epitope solution can contain suitable buffers and the like, including preservatives, antimicrobial agents, stabilizers and the like, as known in the art. The epitope can be in preserved form, such as desiccated, freeze-dried and so on. The epitopes can placed on a suitable solid phase for use in a particular assay. Thus, the epitopes can be placed, and dried, in the wells of a culture plate, spotted on a membrane in a layered array or lateral flow immunoassay device, spotted onto a slide or other support for a microarray, and so on. The items can be packaged as known in the art to ensure maximal shelf life, such as with a plastic film wrap or an opaque wrap, and boxed. The assay container can contain as well, positive and negative control samples, each in a vessel, which includes, when a sample is a liquid, a vessel with a dropper or which has a cap that enables the dispensing of drops, sample collection devices, other liquid transfer devices, detector reagents, developing reagents, such as silver staining reagents and enzyme substrate, acid/base solution, water and so on. Suitable instructions for use may be included.

In other formats, such as using a bead-based assay, the plural epitopes can be affixed to different populations of beads, which then can be combined into a single reagent, ready to be exposed to a patient sample.

The invention now will be exemplified in the following non-limiting examples, which data have been reported in Zhong et al., Am. J. Respir. Crit. Care Med., 172:1308-1314, 2005 and Zhong et al., J. Thoracic Oncol., 1:513-519, 2006, the contents of which are incorporated by reference herein, in entirety.

### EXAMPLES

### Example 1

### NSCLC Diagnostic Assay

In this Example, identification of markers for diagnosing later stage (II, III and IV) NSCLC was undertaken. Two T7 phage NSCLC libraries were biopanned with NSCLC patient and normal plasma to enrich for a population of immunogenic clones expressing polypeptides recognized by antibody circulating in NSCLC patients.

One T7 phage NSCLC cDNA library was purchased (Novagen, Madison, Wis., USA) and a second library was constructed from the adenocarcinoma cell line NCI-1650 using the Novagen OrientExpress cDNA Synthesis and Cloning systems. The libraries were biopanned with pooled plasma from 5 NSCLC patients (stages 2-4; diagnosis confirmed by histology) and from normal healthy donors, to enrich the population of phage-expressed proteins recognized by tumor-associated antibodies. Briefly, the phage displayed library was affinity selected by incubating with protein G agarose beads coated with antibodies from pooled normal sera (250 μl pooled normal sera, diluted 1:20, at 4° C. o/n) to remove non-tumor specific proteins. Unbound phage were separated from phage bound to antibodies in normal plasma by centrifugation. The supernatant then was biopanned against protein G agarose beads coated with pooled patient plasma (4° C. o/n) and separated from unbound phage by centrifugation. The bound/reactive phage were eluted with 1% SDS and then collected by centrifugation. The phage were amplified in E coli NLY5615 (Gibco BRL Grand Island, N.Y.) in the presence of 1 mM IPTG and 50 μg/ml carbenicillin until lysis. Amplified phage-containing lysates were collected and subjected to three additional sequential rounds of biopan enrichment. Phage-containing lysates from the fourth biopan were amplified, individual phage clones were isolated then incorporated into protein arrays as described below.

### Array Construction and High-Throughput Screening

Phage lysates from the fourth round of biopanning were amplified and grown on LB-agar plates covered with 6% agarose for isolating individual phage. A colony-picking robot (Genetic QPix 2, Hampshire, UK) was used to isolate 4000 individual colonies (2000/library). The picked phage were amplified in 96-well plates, then 5 nl of clear lysate from each well were robotically spotted in duplicate on FAST slides (Schleicher and Schuell, Keene, N.H.) using an Affymetrix 417 Arrayer (Affymetrix, Santa Clara, Calif.).

The 4000 phage then were screened with five individual NSCLC patient plasmas not used in the biopan to identify immunogenic phage. Rabbit anti-T7 primary antibody (Jackson Immuno-Research, West Grove, Pa.) was used to detect T7 capsid proteins as a control for phage amount. Both pre-absorbed plasma (plasma:bacterial lysate, 1:30) samples and anti-T7 antibodies were diluted 1:3000 with 1×TBS plus 0.1% Tween 20 (TBST) and incubated with the screening slides for 1 hr at room temperature. Slides were washed and then probed with Cy5-labeled anti-human and Cy3-labeled anti-rabbit secondary antibodies (Jackson ImmunoResearch; 1:4000 each antibody in 1×TBST) together for 1 hr at room temperature. Slides were washed again and then scanned using an Affymetrix 428 scanner. Images were analyzed using GenePix 5.0 software (Axon Instruments, Union City, Calif.). Phage bearing a Cy5/Cy3 signal ratio greater than 2 standard deviations from a linear regression were selected as candidates for use on a “diagnostic chip.”

### Diagnostic Chip Design and Antibody Measurement

Two hundred twelve immunoreactive phage identified in the high-throughput screening above, plus 120 “empty” T7 phage, were combined, re-amp lified and spotted in duplicate onto FAST slides as single diagnostic chips. Replicate chips were used to assay 40 late stage NSCLC samples using the protocol described for screening above. Median of Cy5 signal was normalized to median of Cy3 signal (Cy5/Cy3 signal ratio) as the measurement of human antibody against a unique phage-expressed protein. To compensate for chip to chip variability, measurements were further normalized by subtracting background reactivity of plasma against empty T7 phage proteins and dividing the median of the T7 signal [(Cy5/Cy3 of phage)−(Cy5/Cy3 of T7)/(Cy5/Cy3 of T7)].

Student t-test of normalized signal from 40 patients (stage II-IV) and 41 normals afforded a statistical cutoff (p<0.01) that suggested relative predictive value of each candidate marker. Of the 212 candidates, 17 met that cutoff criterion (p=0.00003 to P=CO1). Redundancy within the group was assessed by PCR and sequence analysis revealing several duplicate and triplicate clones. When redundant clones were eliminated, a set of 7 phage-expressed proteins was identified.

### Statistical Analysis

Logistic regression analysis was performed to predict the probability that a sample was from an NSCLC patient. A total of 81 patient and normal samples were divided into 2 groups. The patients were diagnosed at Stages II-IV of NSCLC. The first group consisted of randomly chosen 21 normal and 20 patient plasma samples which was used as a training set to identify markers that were distinguished between the patient samples and normal samples using individual or a combination of markers. The second group consisting of 20 patient and 20 normal samples was used to validate the prediction rate of the markers identified using the training group. Receiver operating characteristics (ROC) curves were generated to compare the predictive sensitivity and specificity with different markers, and the area under the curve (AUC) was determined. The classifiers were further examined using leave-one-out cross-validation. Smoking history and stage of disease were also analyzed and compared. Then the two groups were reversed, and the group of 40 became the training group to identify markers that were indicative of presence of NSCLC. The markers so identified as providing maximal predictive power then were used to diagnose NSCLC in the other group of 41 samples.

TABLE 5
Areas under the ROC curves and predictive accuracy

| | Training Set* | | | Validation Set † | |
|---|---|---|---|---|---|
| Phange | | Specific- | Sensitiv- | Specific- | Sensitiv- |
| Clone | AUC§ | ity, % | ity, % | ity, % | ity, % |
| 1864 | .857 | 75 | 81 | 65 | 85 |
| 1896 | .857 | 70 | 86 | 70 | 75 |
| 1919 | .824 | 75 | 81 | 70 | 90 |
| 1761 | .798 | 70 | 81 | 70 | 85 |
| 1747 | .864 | 70 | 86 | 70 | 80 |
| 5 Combined | .983 | 92 | 95 | 90 | 95 |

*Training Set consisted of 21 normal and 20 NSCLC patient samples.
† Validation Set consisted of 20 normal and 20 NSCLC patient samples.
§AUC: area under the ROC curve.

TABLE 6
Leave-one-out validation*

| Phange | | | Diagnostic |
|---|---|---|---|
| Clone | Specificity, % | Sensitivity, % | Accuracy†, % |
| 1864 | 70 | 82.9 | 76.5 |
| 1896 | 70 | 82.9 | 75.3 |
| 1919 | 70 | 82.9 | 76.5 |
| 1761 | 60 | 82.9 | 71.6 |
| 1747 | 72.5 | 82.9 | 77.8 |
| 5 Combined | 87.5 | 90.2 | 88.9 |

*Leave-one-out validation: one sample was removed from the testing set containing a total of 81 samples, a classifier was generated for predicting the status (normal or patient) of the removed sample using the rest of the samples. This procedure was repeated for all samples.
†Diagnostic accuracy = (number of true positive + number of true negative)/total number of samples.

### Sequence Analysis of Phage-Expressed Proteins

The 17 phage that were chosen for putative predictive value using the t-test and p value <0.01 were sequenced to identify redundancy, which revealed 7 unique sequences. Although the identity of the phage-expressed proteins is not critical for use in a diagnostic assay of interest, the sequences were compared to those obtained in previous studies that used different (independent) screening methodology and also were compared to the GenBank database to obtain possible identity. Nucleotide sequences obtained from the 7 clones showed homology to GAGE 7, NOPP 140, EEFIA, PMS2L15, SEC15L2, paxillin and BAC clone RP1 1-499F19.

Of the 7 proteins, EEF1A (eukaryotic translation elongation factor 1), a core component of the protein synthesis machinery, and GAGE7, a cancer testis antigen, are overexpressed in some lung cancers. Paxillin is a focal adhesion protein that regulates cell adhesion and migration. Aberrant expression and anomalous activity of paxillin has been associated with an aggressive metastatic phenotypic in some malignancies including lung cancer. PMS2L is a DNA mismatch repair-related protein but no mutation has yet been identified in cancer. Similarly, SEC15L2, an intracellular trafficking protein, and NOPP 140, a nucleolar protein involved in regulation of transcriptional activity, do not have known malignant association. The physiologic function of those three proteins, however, suggests each could have a role in the malignant phenotype.

### Statistical Modeling and Assay Prediction Accuracy

To develop classifiers using the unique 7 phage expressed proteins for higher predictive rates, the 81 samples were divided randomly into two groups, one was used for training purposes and the other for validation. Logistic regression was used to calculate the sensitivity and specificity for predictive accuracy using individual phage expressed proteins as well as a combination of multiple phage expressed markers. Results show that 5 phage markers had significant ability to distinguish patient samples from normal controls in the training set. The ROC AUC for each individually ranged from 0.79 to 0.86. A combination of the 5 markers achieved a promising prediction rate (AUC=0.98), with 95% sensitivity and 85% specificity (Table 5).

Using that statistical model to test the validation group consisting of 20 control normals and 20 NSCLC samples, the assay provided a sensitivity of 90%, and a specificity of 95% (Table 5).

To further examine the association of the classifiers with diagnostic sensitivity and specificity, class prediction using leave-one-out cross-validation on all 81 chips was performed.

Sensitivity and specificity were 90% and 87%, respectively, with the 81 samples, and the overall diagnostic accuracy was 89% (Table 6). Also using all 81 samples, the corresponding clone ID, gene name and p value were as follows: 1864, GAGE7, p=9.1×10″⁹; 1896, BAC clone RP11-499F19, p=3.5×10″⁸; 1919, SEC15L2, p=1.2×10″⁶; 1761, PMS2L15, p=5.2×10″⁷; and 1747, EEFIA, p=5.9×10′⁷. All 5 markers passed a Bonferroni correction of 0.001/262=3.8×10″⁶ making the probability of one or more of them being false positive of less than 0.001.

Therefore, overall, the panel of five markers was used to segregate samples from 40 NSCLC patients and 41 normals with an 89% rate of successful identification when a sample contained all five markers.

### Example 2

### Detecting Early Stage Lung Cancer

In this example, the ability of the assay and method according to the present invention to identify markers able to distinguish stage I lung cancer and occult disease from risk-matched control samples was investigated.

### Human Subjects

Following informed consent, plasma samples were obtained from individuals with histology confirmed NSCLC at the University of Kentucky and Lexington Veterans Administration Medical Center. Non-cancer controls were randomly chosen from 1520 subjects participating in the Mayo Clinic Lung Screening Trial. Briefly, individuals were eligible for the CT screening trial with a minimum 20 pack-year smoking history, age between 50-75, and no other malignancy within five years of study entry, hi addition to non-cancer samples from the Mayo Lung Screening Trial, six stage I NSCLC samples and 40 pre-diagnosis samples were available for analysis. Pre-diagnosis samples were drawn at study entry from subjects diagnosed with NSCLC incidence cancers on CT screening one to five years following sample donation.

### Phage Library

The phage libraries, panning and screening were as described above. Diagnostic chip design and antibody measurement

Two hundred twelve immunoreactive phage identified in the high-throughput screening above, plus 120 “empty” T7 phage, were combined, re-amp lified and spotted in duplicate onto FAST slides as single diagnostic chips. Replicate chips were used to assay 23 stage I NSCLC and 23 risk-matched plasma samples using the protocol described for screening above.

### Statistical Analysis

Normalized Cy5/Cy3 ratio for each of the 212 phage-expressed proteins was independently analyzed for statistically significant differences between 23 patient and 23 control samples by t-test using JMP statistical software (SAS, Inc., Cary, N.C.) as described in the previous example. All 46 samples were used to build up classifiers that were able to distinguish patient from normal samples using individual, or a combination of markers. ROC curves were generated to compare the predictive sensitivity, specificity, and AUC was determined. The classifiers then were examined using leave-one-out cross-validation for all the 46 samples.

The set of classifiers then was used to predict the probability of disease in an independent set of 102 cases and risk-matched controls from a Mayo Clinic Lung Screening Trial. Relative effects of smoking and other non-malignant lung disease were also assessed.

The ROC AUC for each individual marker, achieved by assaying all the 46 samples to estimate predictive ability, ranged from 0.74 to 0.95; and the combination of five markers indicated significant ability to distinguish early stage patient samples from risk-matched controls (AUC=O0.99). The computed sensitivity and specificity using leave-one-out cross-validation were 91.3% and 91.3% respectively (Table 7).

A sample cohort from the Mayo Clinic CT Screening trial that included 46 samples drawn 0-5 years prior to diagnosis (6 prevalence cancers and 40 pre-cancer samples) and 56 risk-matched samples from the screened population was then analyzed as an independent data set. The results indicated accurate classification of 49/56 noncancer samples, 6/6 cancer samples drawn at the time of radiographic detection on a screening CT, 9/12 samples drawn one year prior to diagnosis, 8/11 drawn two years prior, 10/11 drawn 3 years prior, 4/4 drawn four years prior to diagnosis, and 1/2 drawn five years prior to diagnosis, corresponding to 87.5% specificity and 82.6% sensitivity. Three of the eight pre-cancer samples incorrectly classified by the assay had bronchoalveolar cell histology.

In the testing sets, 6/6 non-cancer controls were properly identified with a clinical diagnosis of chronic obstructive pulmonary disease (COPD), one individual with sarcoidosis and one individual with an interval diagnosis of breast cancer. In the latter independent testing set, two individuals with localized prostate cancer were also correctly classified as normal. One individual with a previous diagnosis of breast cancer (>5 years prior) was classified as non-cancer, but a second was classified as cancer. Thirty-four of seventy-nine non-cancer subjects had benign nodules detected on screening CT scans. History of active versus former smoking did not appear to affect predictive accuracy of the test. There was also no association of assay sensitivity with time to diagnosis.

### Sequence Analysis of Phage-Expressed Proteins

The nucleotide sequences of the five predictive phage-expressed proteins were compared to the GenBank database. Nucleotide sequences obtained from the 5 clones used in the final predictive model showed great homology to paxillin, SEC15L2, BAC clone RP1 1-499F19, XRCC5 and MALAT1. The first three were identified as immunoreactive with plasma from patients with advanced stage lung cancer described in the previous example. XRCC5 is a DNA repair gene over-expressed in some lung cancers. Anomalous activity and aberrant expression of paxillin, a focal adhesion protein, has been associated with an aggressive metastatic phenotype in lung cancer and other malignancies. MALAT1 is a regulatory RNA known to be anomalously expressed in lung cancer.

The potential of the instant assay to complement radiographic screening for lung cancer can be recognized in subsequent validation where combined measures of these five antibody markers correctly predicted 49/56 non-cancer samples from the Mayo Clinic Lung Screening Trial, as well as 6/6 prevalence cancers and 32/40 incidence cancers from blood drawn 1-5 years prior to radiographic detection, corresponding to 87.5% specificity and 82.6% sensitivity.

The initial report of the Mayo Clinic Lung Screening Trial described 35 NSCLC diagnosed by CT alone, one NSCLC detected by sputum cytologic examination alone, and one stage FV NSCLC clinically detected between annual screening scans, corresponding to a 94.5% sensitivity of CT scanning alone. Further, retrospective review following the first annual incidence scan revealed small pulmonary nodules were missed on 26% of the prevalence scans, consistent with significant false negative rates reported in other CT screening trials. The diameter of the retrospectively identified nodules was less than 4 mm in 231 participants (62% of those 375 participants), 4-7 mm in 137 (37%), and 8-20 mm in 6 (2%). As such, the 82.6% sensitivity of autoantibody profiling for NSCLC compares quite favorably to that of CT screening alone, by comparison may perform especially well for small tumors, and represents an unparalleled advance in detection of occult disease. Moreover, the 87.5% specificity of the instant assay well exceeds that of CT scanning, which becomes more important as the percentage of benign pulmonary nodules increases in the at-risk population, rising to levels of 69% of participants in the Mayo Clinic Screening Trial.

TABLE 7
Logistic regression and leave-one-out validation in training group

| | Training Set* | | | Validation Set † | |
|---|---|---|---|---|---|
| Phange | | Specific- | Sensitiv- | Specific- | Sensitiv- |
| Clone | AUC§ | ity, % | ity, % | ity, % | ity, % |
| L1919 | 0.85 | 82.6 | 78.3 | 82.6 | 60.9 |
| L1896 | 0.95 | 87 | 87 | 87 | 87 |
| G2004 | 0.80 | 82.6 | 65.2 | 82.6 | 65.2 |
| G1954 | 0.74 | 82.6 | 87 | 73.9 | 69.6 |
| G1689 | 0.82 | 82.6 | 65.2 | 82.6 | 65.2 |
| 5 Combined | 0.99 | 100 | 95.7 | 91.3 | 91.3 |

*Training Set consisted of 23 high-risk normal and 23 NSCLC stage-one patient samples.
† Leave-One-Out Validation: Prediction of single sample based on 45 cases and con trolls.
§AUC: area under the ROC curve.

The five markers accurately diagnosed occult and Phase I lung cancer. Presence of the five markers in a subject can and predicted cancer prior to diagnosis using standard methodologies. Circulating antibodies that bind to NSCLC cells are present in patients that currently are diagnosed as negative using available methodologies.

All references cited herein are herein incorporated by reference in entirety.

It will be evident that various modification can be made to the teachings herein without departing from the spirit and scope of the instant invention.

What is claimed is:

1. A method for reducing the number of false positives associated with radiographic screening for lung cancer, comprising:
(a) providing a test to an asymptomatic patient with a smoking history that measures a panel of two or more autoantibody biomarkers demonstrated to distinguish stage I lung cancer and/or occult disease from risk-matched control samples;
(b) obtaining a blood sample from the patient;
(c) measuring the panel of biomarkers in the blood sample;
(d) determining an amount of the measured autoantibodies in the sample;
(e) calculating a probability the patient is positive for lung cancer using a specificity of at least 65% for the panel as a threshold; and,
(f) selecting for radiographic testing those patients positive for lung cancer according to step (e) whereby the number of false positives associated with radiographic screening for lung cancer have been reduced.

2. The method of claim 1, wherein said radiographic screening is by CT scanning or X-ray.

3. The method of claim 1, wherein the patient is at least 50 years of age.

4. The method of claim 1, wherein the patient has a minimum 20 pack year smoking history.

5. The method of claim 1, wherein said patient is a high risk patient without radiographically detectable lung cancer.

6. The method of claim 1, wherein the panel has a positive predictive value of at least 0.70 as measured by area under the curve (AUC) of receiver operating characteristic (ROC) curves

7. The method of claim 1, further comprising measuring at least three biomarkers, at least four biomarkers, at least five biomarkers, at least six biomarkers, at least seven biomarkers, at least eight biomarkers, at least nine biomarkers or at least ten biomarkers.

8. The method of claim 1, wherein the method further comprises quantification of the measured biomarkers.

9. The method of claim 1, wherein the method further comprises measuring the presence or absence of the biomarkers.

10. The method of claim 1, wherein the specificity threshold of step (e) is greater than 70%, greater than 80% or greater than 90%.

11. A method for reducing the number of false positives associated with radiographic screening for lung cancer, comprising:
(a) providing a test to an asymptomatic patient with a smoking history that measures a panel of two or more autoantibody biomarkers with a positive predictive value of at least 0.70 as measured by area under the curve (AUC) of receiver operating characteristic (ROC) curves;
(b) obtaining a blood sample from the patient;
(c) measuring the panel of biomarkers in the blood sample;
(d) determining an amount of the measured autoantibodies in the sample;
(e) calculating a probability the patient is positive for lung cancer using a specificity of at least 65% for the panel as a threshold; and,
(f) selecting for radiographic testing those patients positive for lung cancer according to step (e) whereby the number of false positives associated with radiographic screening for lung cancer have been reduced.

12. The method of claim 11, wherein said radiographic screening is by CT scanning or X-ray.

13. The method of claim 11, wherein said test has been demonstrated in a population of patients to detect both radiographically apparent cancer (>0.5 cm) as well as occult and pre-malignant cancer that is below the limit of radiographic detection.

14. The method of claim 11, wherein the patient is at least 50 years of age or has a minimum 20 pack year smoking history.

15. The method of claim 11, further comprising measuring at least three biomarkers, at least four biomarkers, at least five biomarkers, at least six biomarkers, at least seven biomarkers, at least eight biomarkers, at least nine biomarkers or at least ten biomarkers.

16. A method for reducing the number of false positives associated with radiographic screening for lung cancer, comprising:
(a) providing a test, to an asymptomatic patient with a smoking history, that measures a panel of two or more autoantibody biomarkers with a positive predictive value of at least 0.70 as measured by area under the curve (AUC) of receiver operating characteristic (ROC) curves;
(b) obtaining a blood sample from the patient;
(c) measuring the panel of biomarkers in the blood sample;
(d) determining an amount of the measured autoantibodies in the sample;
(e) calculating a probability the patient is positive for lung cancer using a specificity of at least 80% for the panel as a threshold; and,
(f) selecting for radiographic testing those patients positive for lung cancer according to step (e) whereby the number of false positives associated with radiographic screening for lung cancer have been reduced.

17. The method of claim 16, wherein said radiographic screening is by CT scanning or X-ray.

18. The method of claim 16, wherein said test has been demonstrated in a population of patients to detect both radiographically apparent cancer (>0.5 cm) as well as occult and pre-malignant cancer that is below the limit of radiographic detection.

19. The method of claim 16, wherein the patient is at least 50 years of age or has a minimum 20 pack year smoking history.

20. The method of claim 16, further comprising measuring at least three biomarkers, at least four biomarkers, at least five biomarkers, at least six biomarkers, at least seven biomarkers, at least eight biomarkers, at least nine biomarkers or at least ten biomarkers.
